(19) United States
(12) Patent Application Publication
(10) Pub. No.: US 2011/0189262 A1
(43) Pub. Date: Aug. 4, 2011

(54) Methods and Compositions for Reducing Systemic Toxicity of Vectors

(75) Inventors: Fan Yuan, Durham, NC (US); Yong Wang, Durham, NC (US); Chuan-yuan Li, Chapel Hill, NC (US)

(73) Assignee: DUKE UNIVERSITY, DURHAM, NC (US)

(21) Appl. No.: 11/884,181

(22) PCT Filed: Feb. 13, 2006
(86) PCT No.: PCT/US06/05044

§ 371 Date: Jun. 24, 2008

Related U.S. Application Data
(60) Provisional application No. 60/652,171, filed on Feb. 11, 2005.

(51) Int. Cl. A61K 31/7088; A61K 9/127; A61P 31/12; A61P 43/00; A61P 37/04

(52) U.S. Cl. 424/450; 514/44 R

(57) ABSTRACT

A method for reducing leakage of a delivery formulation from a target tissue via a damaged blood vessel during and after administration of the delivery formulation is provided. The method includes the steps of: (a) providing a delivery formulation comprising a blocking agent and one or more vectors encoding one or more polypeptides, wherein the delivery formulation is in a liquid form; and (b) administering the delivery formulation to a target tissue in a subject, wherein the administering results in the viscosity of the blocking agent increasing to at least about 100 cP, whereby leakage of the delivery formulation from the target tissue either during or after administration of the delivery formulation is reduced.

### CROSS-REFERENCE TO RELATED APPLICATIONS

This application claims the benefit of U.S. Provisional Patent Application Ser. No. 60/652,171, filed Feb. 11, 2005; the disclosure of which is incorporated herein by reference in its entirety.

### GOVERNMENT INTEREST

The presently disclosed subject matter was made with United States Government support under Grant No. BES-99-84062 awarded by the National Science Foundation and Grant No. CA81512 awarded by the National Institutes of Health. The United States Government has certain rights in the presently disclosed subject matter.

### TECHNICAL FIELD

The presently disclosed subject matter relates to compositions and methods of using the same for sequestering a delivery formulation to a target tissue. In particular, the presently disclosed subject matter relates to delivery formulations comprising a vector and a blocking agent, wherein the blocking agent exhibits an increase in viscosity upon administration to a target tissue, thereby sequestering the vector within the target tissue, and methods of using the same.

### BACKGROUND

One of the major challenges in gene therapy is to safely and efficiently deliver therapeutic genes into target cells in vivo. Gene delivery often relies on viral vectors since they possess specific machinery to facilitate DNA transport into the nucleus of cells. However, viral components or transgene products can be toxic to normal cells.

An additional concern is the immune response raised against viral vectors, which can lead to direct destruction of viral vectors and transfected cells as well as development of neutralizing antibodies in the body that can block the same viral vectors subsequently injected (Blair, 2004; Haegel-Kronenberger et al., 2004; Jooss et al., 1998; Zaiss & Muruve, 2005). As a result, expression of therapeutic genes lasts only for a short period of time and efficiency of gene delivery decreases rapidly when the same vectors are injected repeatedly (Pennuelas et al., 2005). To circumvent this problem, investigators in the art have tried to either suppress the immune system or modify the viral vectors to make them less immunogenic (Zaiss & Muruve, 2005).

Different approaches have been developed for reducing toxicity and immunogenicity in normal tissues. One approach is to switch to non-viral vectors, such as cationic liposomes or polymers. Non-viral vectors are often less toxic and immunogenic. However, non-viral vectors have been demonstrated to be, in general, less efficient in vivo than viral vectors. A second approach is to use tissue-targeting viral vectors, which can be achieved through at least two mechanisms. A first is to incorporate specific molecular structures on the vector surface that can bind to unique markers on the plasma membrane of cells or extracellular matrix in tumors. A second is to incorporate specific transcriptional promoters in viral vectors that can be triggered by either endogenous factors or exogenous interventions. In each of these alternative mechanisms of gene delivery using viral vectors, transgene expression can be primarily restricted to target cells or tissues. However, targeted gene delivery requires identification of unique markers in cells and tissues that can capture the vectors, or identification of specific transcriptional mechanisms that can control gene expression. Both requirements cannot always be achieved.

A third approach to reducing toxicity and immunogenicity is to infuse/inject viral vectors directly into tumors, which can also simultaneously improve interstitial transport of viral vectors in target tissues through at least three mechanisms. One is to establish a pressure gradient for enhancing convection, which is critical for delivery of macromolecules and nanoparticles (e.g., viral vectors). The second is to increase the pore size in tumor tissues due to pressure-induced tissue deformation. Tissue deformation will also, as the third mechanism, improve the connectedness of interstitial space. Thus, direct injection/infusion of vectors is still a desirable option for delivering nucleic acids to target tissue.

It has been widely assumed that the injected vectors should remain localized at the injection site with few injected vector particles escaping from the target tissues. As a result, the direct injection/infusion method should cause less toxicity, compared with the systemic administration of viral vectors. Further, viral vectors should be minimally immunogenic after direct injection/infusion if they do not escape from tumors. However, several research groups have shown that toxicity in normal tissues and immune responses mounted against the vectors are still major concerns in viral gene delivery. This is due primarily to the dissemination of viral vectors beyond the injection site.

Detection of viral dissemination has been facilitated with more sensitive systems using fluorescence and luminescence reporter genes, such as enhanced green fluorescence protein (EGFP) and luciferase. At the cellular and tissue level, EGFP expression was detected both in the liver and tumor tissues, whereas whole body imaging systems, which trace and map the luciferase expression, again revealed that liver was the major disseminating site. It has been estimated that the amount of disseminated viruses outside a tumor can be up to 10 fold higher than what is retained inside tumor tissues, thus reducing the actual ratio of efficacy/toxicity (Wang et al., 2003 and 2005b).

Dissemination can cause serious adverse effects once it occurs. Some mice die within a few hours after intratumor injection, presumably due to vector toxicity resulting from vector dissemination. In non-human primates, it was shown that systemic delivery of high doses of adenoviral vector, resulted in acute toxicity in baboons consistent with activation of the innate inflammatory response, the severity of which was dose dependent, but independent of viral gene expression (Brunetti-Pierri, et al., 2004).

In addition to animal models, toxicity from systemic adenoviral vector exposure is a major obstacle in clinical trials. Autopsy of a patient who soon died after adenovirus treatment showed that liver is a major target tissue for adenovirus (Marshall, 1999). Dissemination causes significant toxicity, which prevents application of higher doses of vector and decreases the amount of vector targeting a tumor, both of which result in reduction of desired clinical efficacy. Disseminated viruses can cause severe acute immune responses to normal tissues and accumulation of cytotoxic transgene products in normal tissues. The disseminated viruses can also stimulate the immune system to develop immunity against virus vectors, preventing effective administration of subsequent doses of vectors.

Therefore, there is presently an unmet need for a gene therapy approach that permits direct injection/infusion of a vector to a target site while also reducing dissemination of the vector from the target site after injection/infusion.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides a method for reducing leakage of a delivery formulation from a target tissue into non-target tissues via a damaged blood vessel during and after administration of the delivery formulation to the target tissue. In some embodiments, the method comprises providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP, whereby leakage of the delivery formulation from the target tissue either during or after administration of the delivery formulation to the target tissue is reduced.

The presently disclosed subject matter further provides a method for decreasing systemic toxicity of a delivery formulation. In some embodiments, the method comprises providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP, whereby systemic toxicity of the delivery formulation is decreased.

The presently disclosed subject matter also provides a method for reducing immune system detection of a delivery formulation in a subject. In some embodiments, the method comprises providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP, whereby immune system detection of the delivery formulation in the subject is decreased.

The presently disclosed subject matter still further provides a method for increasing efficiency of vector delivery to a target tissue. In some embodiments, the method comprises providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP, wherein the increase in viscosity of the blocking agent reduces leakage of the vector from the target tissue to thereby increase the efficiency of vector delivery to the target tissue. In some embodiments, the increased efficiency of vector delivery results in an increase in the expression of the therapeutic polypeptide in the target tissue of at least about 2- to about 4-fold when compared to the expression of the therapeutic polypeptide in the target tissue when an equivalent dosage of the viral vector is administered in the absence of the blocking agent.

In some embodiments of the methods disclosed herein, the vector is a viral vector. In some embodiments, the viral vector is a retrovirus vector, an adenovirus vector, or an adenovirus-associated vector. In some embodiments the vector is a non-viral vector. In some embodiments, the non-viral vector is a plasmid, a bacterium, a water-oil emulsion, a polyethylene imine, a dendrimer, a micelle, a microcapsule, a liposome, or a cationic lipid.

In some embodiments of the methods, the polypeptide is a therapeutic polypeptide. In some embodiments the therapeutic peptide is selected from the group consisting of reporter molecules, immunostimulatory molecules (e.g., a cytokine), enzymes that can convert prodrugs to drugs, tumor suppressor gene products, tumor antigens, apoptosis mediators, toxins, and anti-angiogenic factors.

In some embodiments of the methods, the viscosity of the blocking agent is less than about 20 cP before administration, and increases to at least about 100 cP after administration, and in some embodiments the viscosity of the blocking agent increases to about 200 cP to about 100,000 cP after administration, depending on the shear rate of the solution. In some embodiments, the viscosity of the blocking agent increases to at least about 100 cP within about 1 sec after injection.

In some embodiments of the methods, the blocking agent comprises a poloxamer-containing formulation, which in some embodiments is present in a concentration ranging from about 10% to about 40% by weight. In some embodiments, the blocking agent comprises an alginate-containing formulation, which in some embodiments is present in a concentration ranging from about 0.1% to about 8% by weight.

In some embodiments of the methods, the increase in viscosity of the blocking agent does not result in a significant trapping of the vector in the delivery formulation or a significant reduction in the efficiency of the vector to infect the target tissue.

Accordingly, it is an object of the presently disclosed subject matter to provide methods and compositions for sequestering vectors within a target tissue after injection/infusion of the vectors into the target tissue. This object is achieved in whole or in part by the presently disclosed subject matter.

An object of the presently disclosed subject matter having been stated above, other objects and advantages will become apparent to those of ordinary skill in the art after a study of the following description of the presently disclosed subject matter and non-limiting examples.

### BRIEF DESCRIPTION OF THE DRAWINGS

FIG. 1 is a graph showing the effect of shear rate on viscosity of poloxamer 407 solution at different weight concentrations (i.e., 12.5%, shaded triangles; 15%, open triangles; and 21%, shaded circles) and at a constant temperature of 37° C. The viscosity was measured, using a Brookfield DV-III Rheometer (Brookfield, Mass., U.S.A.). The shear rate was varied from 7.5 to 1875 (1/sec). The standard deviation for each data point is smaller than the size of symbols, and therefore is not shown.

FIGS. 2A-2C are a series of photographs of mice showing luciferase distribution at 24 hr after AdCMVLuc infusion into different tumors: (FIG. 2A) 4T1 in a Balb/c mouse; (FIG. 2B) B16.F10 in a C57BL/6 mouse; and (FIG. 2C) IMR-32 in a Balb/c nude mouse. The dose of infusion was 2.0×10⁸ pfu/tumor.

FIGS. 3A and 3B are a series of photomicrographs showing EGFP expression in 293 cells transfected by AdCMVEGFP that was isolated from plasma (FIG. 3A) and lysed blood cells in mice (FIG. 3B). The blood samples were collected at different time points after the intratumoral infusion of AdCMVEGFP was started. The dose of infusion was 3.0×10⁸ pfu/tumor. The infusion was performed at 1 μl/sec over a 50-sec period.

FIGS. 4A and 4B are a series of photographs of mice showing luciferase distribution in three mice at 10 min (FIG. 4A) and 24 hrs (FIG. 4B) after intravenous injection of 4T1 cells expressing luciferase. The dose of injection was 10⁶ cells/mouse.

FIG. 5 is a series of photomicrographs showing EGFP distribution in different tissues at 24 hrs after the infusion of AdCMVEGFP into 4T1 tumors. Bar=100 μm. The dose of infusion was 3.0×10⁸ pfu/tumor.

FIG. 6 is a bar graph showing the ratio of virus copy numbers between liver and tumor at 10 min after the infusion of AdCMVEGFP into 4T1 tumors implanted in 10 different mice.

FIGS. 7A and 7B show time dependence of bioluminescence intensity in the body after intratumoral infusion of AdCMVLuc.

FIG. 7A is a graph showing bioluminescence intensities in liver (open circles) and tumor (shaded diamonds) at different time points. The intensities were normalized by the values measured at one day post intratumoral infusion of AdCMVLuc. The infusion dose, volume, and rate were 2.0×10⁸ pfu/tumor, 50 μd/tumor, and 1.0 μl/sec, respectively. The symbols represent the means from five animals during the first seven days and from three animals on Day 21. The error bar represents the standard error.

FIG. 7B is a pair of pseudo-color images of luciferase expression in a mouse at 21 days after the intratumoral infusion.

FIG. 8A is a graph showing effects of infusion rate on luciferase expression in the liver (open bars) and tumor (shaded bars). The bioluminescence intensity was determined at two days after intratumoral infusion of AdCMVLuc. The infusion dose and volume were fixed at 2.0×10⁸ pfu/tumor and 50 μl/tumor, respectively. The bars represent the means from five animals and the error bar represents the standard error.

FIG. 8B is a series of digital images showing effects of infusion rate on distributions of blue dye in tumors after intratumoral infusion of Evans blue-labeled albumin solution.

FIG. 9 is a graph showing effects of infusion volume on luciferase expression in the liver (open bars) and tumor (shaded bars). The bioluminescence intensity was determined at two days after intratumoral infusion of AdCMVLuc. The infusion dose and rate were fixed at 2.0×10⁸ pfu/tumor and 1.0 μl/sec, respectively. The bars represent the means from five animals and the error bar represents the standard error. *P<0.05.

FIGS. 10A and 10B are graphs showing effects of infusion dose on luciferase expression in the liver (open bars) and tumor (shaded bars).

FIG. 10A shows bioluminescence intensity determined at two days after intratumoral infusion of AdCMVLuc. The infusion rate and volume were fixed at 1.00/sec and 50 μl/tumor, respectively. The bars represent the means from three to five animals and the error bar represents the standard error. *P<0.05.

FIG. 10B shows bioluminescence intensities of FIG. 10A normalized by the corresponding dose of infusion. The unit of the vertical axis is (10⁵ p/s)/(10⁸ pfu/tumor).

FIGS. 11A and 11B show effects of a poloxamer solution on the amount of adenoviral vectors in the blood, liver, and tumor after the intratumoral infusion of AdCMVEGFP.

FIG. 11A is a pair of photomicrographs (left panel, control; right panel, poloxamer) showing 293 cells treated in vitro by plasma harvested from mice immediately after the intratumoral infusion of AdCMVEGFP. The transfected cells are indicated by the white spots.

FIG. 11B is a graph showing the ratio of copy numbers of adenoviral vectors between tumor and liver tissues harvested at 10 min after the infusion. The bars represent data from individual animals (shaded bars, control; open bars, poloxamer).

FIGS. 12A-12D show the effects of poloxamer solution on the transgene expressions in liver and tumor tissues, respectively.

FIG. 12A is a composite photomicrograph showing EGFP expression, indicated by the white spots, in liver (left panels) and tumor (right panels) tissues after intratumoral infusion of AdCMVEGFP (upper panels, control; lower panels, poloxamer).

FIG. 12B is a pair of photographs of mice (upper rows, control; lower rows, poloxamer) showing bioluminescence distributions in Balb/c mice after intratumoral infusion of AdCMVLuc.

FIG. 12C is a graph showing the average bioluminescence intensities in the liver and the tumor (N=5 in each group; shaded bars, control; open bars, poloxamer).

FIG. 12D is a graph showing ratios of bioluminescence intensities between the tumor and the liver. The bars represent data from individual animals in FIG. 12D (shaded bars, control; open bars, poloxamer). The intensities in FIGS. 12C and 12D were measured directly from the bioluminescence images shown in FIG. 12B.

FIGS. 13A-13D show effects of the poloxamer solution on the convective transport in the tail vein of Balb/c mice. Suspension of rhodamine-labeled nanospheres or AdCMVLuc with or without poloxamer was injected into the tail vein over a 5-second period. The volumes of all injections were 50 and the dose of AdCMVLuc was 1.0×10⁸ pfu/mouse. The concentration of poloxamer was 21%. Fluorescence images of the tail at different time points (0, 2 sec, 5 sec, and 10 sec) after injection of nanospheres suspended in PBS and poloxamer solution are shown in FIGS. 13A and 13B, respectively. Bioluminescence distribution in mice at 24 hrs after the injection of AdCMVLuc suspended in PBS and the poloxamer solution are shown in FIGS. 13C and 13D, respectively.

### BRIEF DESCRIPTION OF THE SEQUENCE LISTING

SEQ ID NOs:1 and 2 are forward and reverse nucleic acid primer sequences for PCR amplification of the adenovirus E4 region, respectively.

SEQ ID NO:3 is a nucleic acid sequence probe for capture of adenovirus nucleic acids.

### DETAILED DESCRIPTION

Systemic vector dissemination is currently a major challenge in vector-based cancer gene therapy. It can result in at least two problems. First, an undesirable immune response can occur. This is a general problem associated with vectors, including nearly all types of viral vectors developed thus far. The immune system can react to antigens on the vector and develop immunity against the vector, preventing subsequent effective administration of the vector in a subject: The immune system can also stimulate an inflammatory response, either in localized tissue where the vectors reside, or systemically, either of which can be potentially life threatening. A second undesirable problem associated with systemic vector dissemination is systemic expression of cytotoxic genes in normal tissues.

The presently disclosed subject matter provides a liquid delivery formulation that can dramatically inhibit systemic vector dissemination to normal tissues. It can significantly enhance the safety of gene delivery. Further, gene expression in tumors can be increased, at least partly due to the forced localization of the vectors.

The presence of vectors during intratumoral infusion/injection in the blood results from the leakiness of tumor vessels, which can be due to either the vessel damage during needle insertion or large pores occurring naturally in tumor vessels. Although not wishing to be limited by theory, it is believed that the increase in the viscosity of vector suspensions can significantly reduce interstitial fluid flow and blood flow in tumor vessels. Thus, the presently disclosed subject matter provides methods and compositions for inhibiting systemic vector dissemination by blocking blood flow in tumor vessels utilizing a delivery formulation comprising the vector and a blocking agent, which can exhibit increased viscosity when administered to a target tissue.

The inhibition of systemic vector dissemination will in turn increase the retention of vectors in tumor tissues. Consequently, transgene expression in tumors is increased. Further, the blocking agent not only blocks vector dissemination, but also stops blood flow in tumor vessels. The latter can result in direct damage to tumor cells as well, since their survival depends on nutrient supply via blood circulation. Further, local sequestration of the vector reduces systemic immune responses. A reduction in immune response can prolong the expression of therapeutic genes in tumors and allow repeated vector gene therapy without any vector modification or immunosuppression of the subject's immune system. Finally, reduced dissemination of vectors from the injection site can limit systemic toxicity and healthy tissue damage resulting directly from vector infection and indirectly from expression of viral gene products.

The compositions and methods of the presently disclosed subject matter provide several advantages over alternative solutions, such as encapsulation of vectors in solid polymeric devices, such as nanoparticles, microspheres, discs, films, and rods prior to gene delivery. Release of vectors from these devices is inefficient and diffusion of vectors from these devices to deeper layers of tumor tissues is very slow. For example, diffusion of viral particles with a diameter of 100 nm over a distance of 100 μm will take several days. During this period, the viral vectors can lose their effectiveness before even reaching target cells.

### I. DEFINITIONS

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a vector” includes a plurality of such vectors, and so forth.

The term “about”, as used herein when referring to a measurable value such as an amount of weight, time, etc. is meant to encompass variations of in some embodiments ±20% or ±10%, in some embodiments ±5%, in some embodiments ±1%, in some embodiments ±0.5%, and in some embodiments ±0.1% from the specified amount, as such variations are appropriate to perform the disclosed methods.

As used herein, the term “blocking agent” refers to a composition that changes in viscosity when introduced into an environment within a subject. In some embodiments, the change in viscosity results from the environment in the subject being of a different pH (e.g., lower) than the pH or a different temperature (e.g., higher) of the delivery formulation comprising the blocking agent.

The term “gene expression” generally refers to the cellular processes by which a polypeptide is produced from a DNA sequence present within a vector and exhibits an activity in a cell. In some embodiments, the polypeptide exhibits a therapeutic activity. As such, gene expression involves the processes of transcription and translation, but also involves post-transcriptional and post-translational processes that can influence an activity of a gene or gene product. These processes include, but are not limited to RNA syntheses, processing, and transport, as well as polypeptide synthesis, transport, and post-translational modification of polypeptides. Additionally, processes that affect the entry of a vector into a target cell can also affect gene expression as defined herein.

As used herein, the terms “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally occurring nucleotides (such as deoxyribonucleotides and ribonucleotides), or analogs of naturally occurring nucleotides (e.g., α-enantiomeric forms of naturally-occurring nucleotides), or a combination of both. Nucleic acids can be either single stranded or double stranded. In some embodiments, a viral vector comprises a nucleic acid sequence encoding a therapeutic polypeptide.

As used herein, the term “polypeptide” means any polymer comprising any of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably. The term “polypeptide” encompasses proteins of all functions, including enzymes.

In some embodiments, a polypeptide encoded or delivered by a vector to a target tissue can include therapeutic polypeptides. The phrase “therapeutic polypeptide” refers to a polypeptide, wherein provision of the polypeptide to a host cell produces a beneficial effect or aids in the determination of a diagnosis (e.g., an imaging agent or a polypeptide associated with an imaging agent). A beneficial effect can comprise replacement of an abnormally reduced or lost biological activity. A beneficial effect can also comprise antagonism of an abnormally elevated or ectopic biological activity, or even a cytotoxic or cytostatic effect against an abnormal cell (e.g., a tumor cell). In some embodiments, a therapeutic polypeptide is provided to a host cell via a vector (for example, a viral vector) that comprises a nucleotide sequence that encodes the therapeutic polypeptide. Exemplary therapeutic polypeptides include but are not limited to reporter molecules, immunostimulatory molecules (e.g., cytokines, etc.), enzymes that can convert prodrugs to drugs, tumor suppressor gene products, tumor antigens, apoptosis mediators, toxins, and anti-angiogenic factors.

Representative therapeutic proteins with immunostimulatory effects include but are not limited to cytokines (e.g., IL-2, IL-4, IL-7, IL-12, interferons, granulocyte-macrophage colony-stimulating factor (GM-CSF), tumor necrosis factor alpha (TNF-α)), immunomodulatory cell surface proteins (e.g., human leukocyte antigen (HLA proteins), co-stimulatory molecules (e.g., B7.1), and tumor antigens.

A representative enzyme that can convert prodrugs to drugs is, for example, cytosine deaminase (CD), which can convert 5-fluorocytosine (5-FC) (prodrug) to 5-fluorouracil (5-FU) (drug). Another representative enzyme that can convert a prodrug to an active drug and herpes simplex virus thymidine kinase (HSV-TK), which can phosphorylate the prodrugs ganciclovir (GCV) and acyclovir (ACV) to active cytotoxins. Both of these exemplary enzyme/prodrug systems are known in the art for their applicability to cancer gene therapy regimens. However, the recitation of these particular examples is not intended to limit the presently disclosed subject matter to only enzyme/prodrug systems useful for cancer gene therapy applications.

Toxins are generally complex toxic products of various organisms including bacteria, plants, etc. Representative toxins include but are not limited to coagulants such as Russell's Viper Venom, activated Factor IX, activated Factor X or thrombin; and cell surface lytic agents such as phospholipase C, (Flickinger & Trost, 1976) or cobra venom factor (CVF) (Vogel & Muller-Eberhard, 1981) which should lyse neoplastic cells directly. Additional examples of toxins include but are not limited to: ricin, ricin A chain (ricin toxin), Pseudomonas exotoxin (PE), diphtheria toxin (DT), bovine pancreatic ribonuclease (BPR), pokeweed antiviral protein (PAP), abrin, abrin A chain (abrin toxin), gelonin (GEL), saporin (SAP), modeccin, viscumin and volkensin.

The term “angiogenesis” refers to the process by which new blood vessels are formed. The terms “anti-angiogenic response” and “anti-angiogenic activity” as used herein, each refer to a biological process wherein the formation of new blood vessels is inhibited.

Representative proteins with anti-angiogenic activities that can be used in accordance with the presently disclosed subject matter include: thrombospondin I (Kosfeld & Frazier, 1993; Tolsma et al., 1993; Dameron et al., 1994), metallospondin proteins (Carpizo & Iruela-Arispe, 2000), class I interferons (Albini et al., 2000), IL-12 (Voest et al., 1995), protamine (Ingber et al., 1990), angiostatin (O'Reilly et al., 1994), laminin (Sakamoto et al., 1991), endostatin (O'Reilly et al., 1997), and a prolactin fragment (Clapp et al., 1993). In addition, several anti-angiogenic peptides have been isolated from these proteins (Maione et al., 1990; Eijan et al., 1991; Woltering et al., 1991).

The term “substantially identical” in the context of two or more nucleic acid or polypeptide sequences is measured as nucleic acid or polypeptide sequences are in some embodiments about 35%-45%, in some embodiments about 45-55%, in some embodiments about 55-65%, in some embodiments about 65-75%, in some embodiments about 75-85%, in some embodiments about 85-95%, and in some embodiments greater than 95% nucleotide or amino acid identity or similarity (with identity and similarity differing in that the latter allows for substitution of functionally equivalent amino acids or codons encoding functionally equivalent amino acids). In some embodiments, two or more “substantially identical” nucleic acid or polypeptide sequences have about 70%, in some embodiments about 80%, in some embodiments about 90%, in some embodiments about 95%, and in some embodiments about 99% identity or similarity at the nucleotide or amino acid level. Percent “identity” and “similarity”, and methods for determining identity and similarity are known in the art, and include such programs as BLAST (available at the website for the National Center for Biotechnology Information (NCBI)), and the GAP® program that is available from Accelrys, Inc. of San Diego, Calif., as a part of the GCG® WISCONSIN PACKAGE®.

The term “subject” as used herein refers to any invertebrate or vertebrate species. The methods disclosed herein are particularly useful in the treatment of warm-blooded vertebrates. Thus, the presently disclosed subject matter concerns mammals and birds. More particularly, provided is the treatment of mammals such as humans, as well as those mammals of importance due to being endangered (such as Siberian tigers), of economic importance (animals raised on farms for consumption by humans), and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

The term “target tissue” as used herein refers to an intended site for accumulation of a delivery formulation following administration to a subject. In some embodiments of the disclosed methods, the target tissue is present within a vertebrate subject, in some embodiments a mammal, and in some embodiments a human. In some embodiments, the target tissue comprises a tumor or neoplasm, in some embodiments a tumor or neoplasm selected from the group consisting of benign intracranial melanomas, arteriovenous malformation, angioma, macular degeneration, melanoma, adenocarcinoma, malignant glioma, prostatic carcinoma, kidney carcinoma, bladder carcinoma, pancreatic carcinoma, thyroid carcinoma, lung carcinoma, colon carcinoma, rectal carcinoma, brain carcinoma, liver carcinoma, breast carcinoma, ovary carcinoma, solid tumors, solid tumor metastases, angiofibromas, retrolental fibroplasia, hemangiomas, Kaposi's sarcoma, and combinations thereof.

The term “viscosity” as used herein refers to the thickness of a fluid, that is, its resistance to pouring. Viscosity is commonly quantitated as the resistance of a fluid to deformation under sheer stress. Viscosity describes a fluid's internal resistance to flow and may be thought of as a measure of fluid friction. Thus, water is “thin” and has a low viscosity, while oils are “thick” and have a high viscosity. For viscoelastic materials, such as most polymer solutions, their viscosity depends on the shear rate (i.e., the spatial gradient of fluid velocity). To emphasize this dependence, those of ordinary skill in the art sometimes use “apparent viscosity” instead of “viscosity” to refer to the thickness of viscoelastic solutions. In the presently disclosed subject matter, however, the term “viscosity” refers to the thickness of both viscous and viscoelastic solutions. The International System of Units (SI) standard physical unit of viscosity is the Pascal-second (Pa·s), which is identical to 1 N·s/m² or 1 kg/(m·s). However, the centimeter-gram-second system (cgs) physical unit for dynamic viscosity is the poise (P) named and is more commonly used in the art, especially as centipoise (cP). Conveniently, water has a viscosity of 1.0020 cP at 20° C. Therefore, cP is used as the unit of measure to describe viscosity in the present application. For reference, 1 cP=1 mPa·s. Viscosity can be measured by any of several techniques generally known in the art. For example, viscosity can be measured with various types of viscometer, typically at a standard temperature, such as for example 25° C. or 37° C. In particular, a Brookfield DV-III Rheometer (Brookfield, Mass., U.S.A.) was used to determine the viscosity measurement of liquids disclosed herein. The temperature of a water bath can be set to a standard temperature for all measurements, e.g., 37° C. Samples to be measured can be placed in a container in the water bath and the temperature throughout the sample equalized to that of the water bath by incubation for a period of time prior to viscosity measurement. The measurement can be repeated several times, e.g., 3 times and the average value of the measurements used as the reported viscosity measurement.

### II. DELIVERY FORMULATIONS

Although intratumoral injection/infusion is a promising approach for gene therapy treatments of target tissues, including for example tumors, potential problems exist related to systemic dissemination from treated tissue to normal tissues during and after the injection/infusion. The presently disclosed subject matter provides delivery formulations for delivery of vectors to target tissues (e.g., a tumor) that address these issues.

The delivery formulation sequesters a vector (for example, a viral vector) within the target tissue, while still permitting dispersion of the vector within the formulation. This allows dissemination of the vector within the target tissue, while reducing broad dissemination of the vector beyond the target tissue and throughout the subject. As previously discussed, localization of the vector to the target tissue provides several advantages, including a reduction in systemic toxicity, a reduction in any potential generalized immune system response to the vector, and an increase in efficacy of the vector due to an increase in concentration of the vector within the target tissue.

The delivery formulation comprises a vector (for example, a viral vector) and a blocking agent having the advantageous property of a viscosity that substantially increases upon exposure to the target tissue. That is, the viscosity of the blocking agent is low prior to and during administration of the formulation to the target tissue, but increases rapidly upon entry into the target tissue. Thus, the initial low viscosity of the blocking agent allows relative ease of formulation and delivery of the composition to the target tissue, but after delivery, the significantly increased viscosity facilitates sequestration of the vector within the target tissue.

Accordingly, the presently disclosed subject matter provides in some embodiments delivery formulations that increase in viscosity upon administration to a subject in order to retain the vector in the target tissue by preventing leakage of the vector through blood vessels damaged during the administration event, and further provides that the viscosity change does not result in an unacceptable (i.e., a significant) trapping of the vector in the delivery formulation or an unacceptable (i.e., a significant) reduction in the ability of the vector to efficiently infect the target tissue, in the case of a viral vector.

Additional components can also be present in the delivery formulation, including but not limited to pharmaceutically acceptable carriers and excipients. For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents.

The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some preferred ingredients are SOS, for example in the range of 0.1 to 10 mg/ml, preferably about 2.0 mg/ml; and/or mannitol or another sugar, for example in the range of 10 to 100 mg/ml, preferably about 30 mg/ml, and/or phosphate-buffered saline (PBS). The additional components can be mixed with the blocking agent during formulation of the blocking agent and/or viral vector to a suitable concentration, and/or can be added to the delivery formulation prior to administration.

It should be understood that in addition to the ingredients particularly noted above the formulations of the present subject matter can include other agents conventional in the art having regard to the type of formulation in question. Of the possible formulations, sterile pyrogen-free aqueous and non-aqueous solutions are preferred.

The therapeutic regimens and pharmaceutical compositions of the presently disclosed subject matter can be used with additional adjuvants or biological response modifiers as well including, but not limited to, the cytokines IFN-α, IFN-γ, IL2, IL4, IL6, TNF, or other cytokine affecting immune cells. In accordance with this aspect of the presently disclosed subject matter, the disclosed formulations can be administered in combination therapy with one or more of these cytokines or cytokine affecting immune cells.

II.A. Blocking Agents

To reduce the dissemination of vectors beyond target tissues, the presently disclosed subject matter provides delivery formulations comprising a vector mixed with a blocking agent that increases its viscosity after administration to a target tissue, thereby sequestering the vector in the target tissue, and methods of using the delivery formulations. In some embodiments the vector is a viral vector. In some embodiments the vector encodes a polypeptide, for example, a therapeutic polypeptide.

In some embodiments of the presently disclosed subject matter, a delivery formulation comprises a blocking agent having an initial low viscosity which rapidly increases to a higher viscosity after administration to a target tissue. The change in viscosity of the blocking agent can be in response to a change in pre-administration and post-administration environmental conditions. Any liquid formulation whose viscosity is sensitive to environmental factors (e.g., temperature, pH, ionic concentration) can be used to reduce/block systemic vector dissemination from target tissue, including for example solid tumors.

Thus, as used herein, the term “blocking agent” refers to a composition that changes in viscosity when introduced into an environment within a subject. In some embodiments, the change in viscosity is an increase in viscosity from about the viscosity of fluid normally found in the target tissue to at least about 10-, 25-, 50-, 100-, 200-, 250-, 300-, 400-, 500-, or 1000-fold higher than the viscosity of the fluid found in the target tissue. The change in viscosity can occur as a result of exposure of the blocking agent to the temperature, pH, ionic concentration, or any other characteristic of the environment. The environment can be either intrinsic or modified through treatment (e.g., tumor tissues can be heated by external sources in hyperthermia treatment.) In some embodiments, the change in viscosity results from the temperature in the environment being higher than the temperature of the blocking agent prior to its administration into the subject. In some embodiments, the change in viscosity results from the environment in the subject being of a different pH (e.g., lower) than the pH of the delivery formulation comprising the blocking agent.

Representative blocking agents include, but are not limited to poloxamer-based liquid formulations and alginate-containing formulations. Poloxamer block copolymers conforming to the NF 19 specifications are available from BASF Corporation (Mount Olive, N.J., United States of America) under the PLURONIC® registered tradename. The PLURONIC® block copolymers are synthetic copolymers of ethylene oxide and propylene oxide, and have the following general chemical structure:

HO(C₂H₄O)ₐ(C₃H₆O)_{b}(C₂H₄O)ₐH.

In some embodiments, the blocking agent comprises poloxamer selected from the group consisting of poloxamer 188, poloxamer 338, poloxamer 407, and combinations thereof. For poloxamer 188, a=80 and b=27; for poloxamer 407, a=101 and b=56; and for poloxamer 338, a=141 and b=44. It should be noted, however, that the methods and compositions of the presently disclosed subject matter are not limited to the use of these three poloxamers, and that other poloxamer-based formulations, either commercially available or custom synthesized, can also be employed either alone or in combination.

In those embodiments for which viscosity is at least partly dependent on temperature, the resultant viscosity of the delivery formulation can also depend on the blocking agent employed and its concentration in the delivery formulation. For example, the viscosity of poloxamer solutions at different weight concentrations (%) has been examined. Generally, viscosity is dependent on shear rates of the delivery formulation. For various poloxamer-based blocking agents, the viscosity ranges at 37° C. are from 40 cP for a 12% solution to 200-100,000 cP for a 21% solution. At 4° C., the viscosity is typically less than 30 cP for a 21% solution. When the concentration is extremely high, the solution becomes too viscous to be prepared and injected, although a better result in terms of the inhibition of virus dissemination might be expected. Additionally, different poloxamers are available (including, for example, poloxamers 188, 338, and 407). Different poloxamers can be mixed together to produce a blocking agent, which can be used to achieve a concentration that is higher than would otherwise be possible using individual poloxamers (e.g., 21%) without causing significant problems in solution preparation and injection.

Another aspect that can be modified to optimize viscosity changes is the rate of injection of the delivery formulation, because the degree of viscosity change can depend on both the concentration of polymer and the amount of heat transfer into the polymer solution. Thus, each parameter can be adjusted as needed to produce optimal effect. For example, either “low poloxamer concentration and low injection rate” or “high concentration and high injection rate” can achieve the same effects on blocking the virus dissemination.

Accordingly, in some embodiments the effective weight concentration of poloxamer in poloxamer-containing formulations is between about 12% and 40% and the effective weight concentration of alginate in alginate-containing formulations is between about 0.1% and 8%, depending on the rate of injection and the combination of different polymers.

In a particular formulation, it was confirmed the viscosity of the poloxamer solution (21%, w/w) depended on temperature and shear rate. When the temperature was changed from 4° C. to 37° C., the viscosity could be increased by several orders of magnitude, depending on the shear rate. At the shear rate of 7.5 sec⁻¹, the viscosity was increased from 41 to 4200 cp within a few seconds.

Additionally, certain blocking agents that are made up of shear-thinning materials (i.e., compositions that become less viscous when sheared at a high shear stress) can be injected as a gel, which then as a result of the shear-stress associated with the injection process become less viscous (i.e. liquefy), before regaining a gel state when the shear stress is removed (i.e. when deposited in the target tissue). Poloxamer-based blocking agents have this capacity, and thus poloxamer gel and/or a poloxamer solution can be injected into target tissues since poloxamer gel can become a liquid under a high shear stress. Accordingly, the initial viscosity (i.e., before the injection) of certain delivery vehicles can vary from similar to or equal to the viscosity of blood to much higher, so long as the solution can be easily prepared and injected.

In some embodiments, the viscosity of the blocking agent, and thereby the delivery formulation, increases to at least about 100 cP, and in some embodiments to at least about 1,000 cP. In some embodiments, the viscosity increases within about 30 seconds, in some embodiments within about 10 seconds, and in some embodiments within about 1 second or less after injection. In some embodiments, the viscosity of the blocking agent increases to a range of from about 200 cP to about 100,000 cP and in some embodiments, to a range of from about 4,000 cP to about 50,000 cP, depending on the shear rate of the solution.

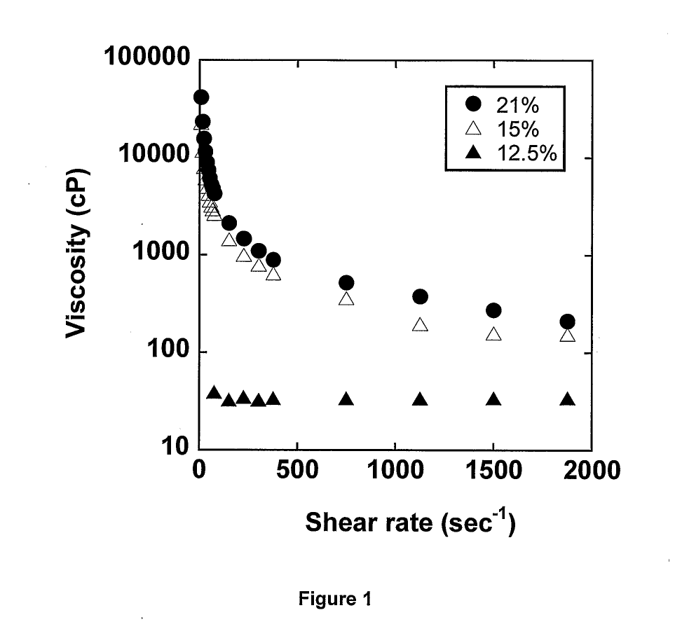

Viscosity can decrease with increasing shear rate (e.g., with increasing rate of intratumoral injection). FIG. 1 is a graph demonstrating how shear rate can affect viscosity. The effect of shear rate on viscosity of a poloxamer 407-containing solution at different concentrations (i.e., 12.5%, 15%, and 21%) at a constant temperature of 37° C. was measured. The shear rate was varied from 7.5 to 1875 (1/sec) for each solution and the measured viscosity for 15% and 21% solutions varied according to the sheer rate changes, as shown in FIG. 1. The rate at which the viscosity change occurs also depends, for example, on the rate of heat transfer, pH change, or ion diffusion into the formulation, which can be faster for a small volume of the formulation and slower for a large volume of the formulation.

In some embodiments, the presence of the blocking agent in the delivery formulation increases the retention of the vector at the administration site, and thus reduces the proportion of vector that enters the circulation while simultaneously enhancing the efficiency of infection of the target tissue with the vector.

In some embodiments, the presence of the blocking agent in the delivery formulation enhances the expression of the therapeutic polypeptide in the target tissue relative to the expression that would be achieved by administering the vector encoding the therapeutic polypeptide in the absence of the blocking agent. In some embodiments including viral vectors the increase in expression can result from either an increase in the number or proportion of cells that get infected by the viral vector, or the level at which individual infected cells express the therapeutic polypeptide.

In a particular embodiment of the presently disclosed subject matter, a biocompatible ABA block copolymer, poloxamer 407, was utilized as a blocking agent. This poloxamer was shown to significantly increase the viscosity of virus suspension when the temperature was changed from 4° C. to 37° C. Using this composition, for example, transgene expression in solid tumors can be significantly increased and virus dissemination can be reduced by two orders of magnitude after intratumoral infusion of adenoviral vectors, as set forth in detail in Example 6.

Without wishing to be bound by theory, it is believed that the mechanism of reduction is due at least in part to the viscous poloxamer solution blocking convection of vectors in the interstitial space and the lumen of microvessels in the vicinity of the infusion site. Thus, these compositions can be used in gene therapy to enhance efficacy, reduce systemic toxicity, and reduce systemic immune response to the vector.

II.B. Vectors

The presently disclosed subject matter includes delivery formulations comprising vectors. The particular vector employed in accordance with the methods of the present subject matter is not intended to be a limitation of the methods. Thus, any suitable vector for delivery of the gene therapy construct can be used.

The vectors can in some embodiments encode therapeutic polypeptides, carry polynucleotides that encode therapeutic polypeptides, or even directly carry therapeutic polypeptides, and can include plasmids, cosmids, bacteria, viral vectors, water-oil emulsions, polyethylene imines, dendrimers, micelles, microcapsules, liposomes, or cationic lipids. Thus, the term “vector”, as used herein refers to a vehicle (e.g., a modified viral particle) that can carry specific nucleic acid sequences (e.g., coding sequences encoding polypeptides) or polypeptides directly and deliver them to cells in target tissues (e.g., tumor cells). In some embodiments, a vector comprises a nucleic acid molecule (e.g., a DNA molecule) having sequences that enable its replication in a compatible host cell. In some embodiments, the vector can also replicate in the target tissue, although in some embodiments the vector is designed such that it cannot replicate in the target tissue. A vector also includes nucleotide sequences to permit ligation of nucleotide sequences within the vector, wherein such nucleotide sequences are also replicated in a compatible host cell. A vector can also mediate recombinant production of a therapeutic polypeptide encoded by the vector.

As noted, the vector can be a viral vector or a non-viral vector. Suitable viral vectors that can be used to deliver nucleic acid molecules to target cells are known in the art, and include, but are not limited to, adenoviruses, adeno-associated viruses (AAVs), retroviruses, pseudotyped retroviruses, herpes viruses, vaccinia viruses, Semliki Forest virus, and baculoviruses. Suitable non-viral vectors comprise plasmids, bacteria, water-oil emulsions, polyethylene imines, dendrimers, micelles, microcapsules, liposomes, and cationic lipids. Polymeric carriers for gene therapy constructs can be used as described in Goldman et al., 1997 and U.S. Pat. Nos. 4,551,482 and 5,714,166. Peptide carriers are described in U.S. Pat. No. 5,574,172. Where appropriate, two or more types of vectors can be used together. For example, a plasmid vector can be used in conjunction with liposomes. Currently, an embodiment of the present subject matter envisions the use of a retrovirus, an adenovirus, or an adeno-associated virus.

As desired, vectors, especially viral vectors, can be selected to infect target cells and in some preferred instances achieve integration of the nucleic acid of a polynucleotide construct encoding a polypeptide, into the genome of the cells to be transformed or transfected. Including a ligand in the complex having affinity for a specific cellular marker can also enhance delivery of the complexes to a target in vivo. Ligands include antibodies, cell surface markers, viral peptides, and the like, which act to home the complexes to tumor vasculature or endothelial cells associated with tumor vasculature, or to tumor cells themselves. A complex can comprise a construct or a secreted therapeutic polypeptide encoded by a construct. An antibody ligand can be an antibody or antibody fragment specific towards a tumor marker such as Her2/neu (v-erb-b2 avian erythroblastic leukemia viral oncogene homologue 2), CEA (carcinoembryonic antigen), ferritin receptor, or a marker associated with tumor vasculature (integrins, tissue factor, or β-fibronectin isoform). Antibodies or other ligands can be coupled to carriers such as liposomes and viruses, as is known in the art. See, e.g., Neri et al., 1997; Kirpotin et al., 1997; Cheng, 1996; Pasqualini et al., 1997; Park et al., 1997; Nabel, 1997; U.S. Pat. No. 6,071,890; and European Patent No. 0 439 095. Alternatively, pseudotyping of a retrovirus can be used to target a virus towards a particular cell (Marin et al., 1997).

Suitable methods for introduction of a gene therapy construct into cells include direct injection or infusion into a cell mass (e.g., target tissue), for example. A delivery method is selected based on considerations such as the vector type, the toxicity of the encoded gene, and the condition to be treated.

Viral vectors of the presently disclosed subject matter can in some embodiments be disabled, e.g. replication-deficient. That is, they lack one or more functional genes required for their replication, which prevents their uncontrolled replication in vivo and avoids undesirable side effects of viral infection. In some embodiments, all of the viral genome is removed except for the minimum genomic elements required to package the viral genome incorporating the therapeutic gene into the viral coat or capsid. For example, it can be desirable to delete all the viral genome except the Long Terminal Repeats (LTRs) or Inverted Terminal Repeats (ITRs) and a packaging signal. In the case of adenoviruses, deletions are typically made in the E1 region and optionally in one or more of the E2, E3 and/or E4 regions. In the case of retroviruses, genes required for replication, such as env and/or gag/pol can be deleted. Deletion of sequences can be achieved by recombinant means, for example, involving digestion with appropriate restriction enzymes, followed by religation. Replication-competent self-limiting or self-destructing viral vectors can also be used.

Nucleic acid constructs of the presently disclosed subject matter can be incorporated into viral genomes by any suitable means known in the art. Typically, such incorporation will be performed by ligating the construct into an appropriate restriction site in the genome of the virus. Viral genomes can then be packaged into viral coats or capsids by any suitable procedure. In particular, any suitable packaging cell line can be used to generate viral vectors of the presently disclosed subject matter. These packaging lines complement the replication-deficient viral genomes of the presently disclosed subject matter, as they include, typically incorporated into their genomes, the genes which have been deleted from the replication-deficient genome. Thus, the use of packaging lines allows viral vectors of the present subject matter to be generated in culture.

Suitable packaging lines for retroviruses include derivatives of PA317 cells, ψ-2 cells, CRE cells, GRIP cells, E-86-GP cells, and 293GP cells. Line 293 cells can be used for adenoviruses and adeno-associated viruses.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Exemplary, non-limiting methods are described in Sambrook & Russell, 2001; Silhavy et al., 1984; Ausubel et al., 1992; and in Glover, 1985. Site-specific mutagenesis to create base pair changes, deletions, or small insertions is also known in the art as exemplified by, for example, Adelman et al., 1983; and Sambrook & Russell, 2001.

II.C. Administration

Suitable methods for administration of a delivery formulation comprising vectors and blocking agents of the present subject matter include but are not limited to subcutaneous, intramuscular, or intratumoral injection/infusion. The particular mode of administering a delivery formulation of the presently disclosed subject matter depends on various factors, including the distribution and abundance of cells to be treated, the vector and/or blocking agent employed, additional tissue- or cell-targeting features of the vector, and mechanisms for metabolism or removal of the vector from its site of administration. For example, relatively superficial tumors can often be injected or infused intratumorally.

Preferably, the method of administration encompasses features for regionalized vector delivery at the target tissue site in need of treatment. In one embodiment, a delivery formulation is delivered intratumorally. When employing intratumoral delivery, the delivery formulation preferably comprises a viral vector encoding a therapeutic polypeptide and a blocking agent.

II.D. Dose

A therapeutically effective amount of a vector encoding one or more therapeutic polypeptides of the presently disclosed subject matter is administered to a subject in need thereof. A “therapeutically effective amount” is an amount of the vector sufficient to produce a measurable response (e.g., a cytotoxic, immunostimulatory or anti-angiogenic response within the target tissue of a subject being treated). In one embodiment, an activity that inhibits tumor growth is measured. Actual dosage levels of vectors in the pharmaceutical compositions of the presently disclosed subject matter can be varied so as to administer an amount of the therapeutic polypeptide(s) that is effective to achieve the desired therapeutic response for a particular subject. The selected dosage level will depend upon the mechanism of action and activity of the therapeutic polypeptide, the route of administration, combination with other drugs or treatments, the severity of the condition being treated, and the condition and prior medical history of the subject being treated. However, it is within the skill of the art to start doses of the vector encoding the therapeutic polypeptide at levels lower than required to achieve the desired therapeutic effect and to gradually increase the dosage until the desired effect is achieved.

The potency of a therapeutic polypeptide can vary, and therefore a “therapeutically effective” amount of a vector encoding the polypeptide can vary. However, using the assay methods described herein below, one skilled in the art can readily assess the potency and efficacy of a therapeutic polypeptide and adjust the therapeutic regimen accordingly.

After review of the disclosure herein of the present subject matter, one of ordinary skill in the art can tailor the dosages to an individual subject, taking into account the particular formulation, method of administration to be used with the composition, and tumor size, for example. Further calculations of dose can consider patient height and weight, severity and stage of symptoms, and the presence of additional deleterious physical conditions. Such adjustments or variations, as well as evaluation of when and how to make such adjustments or variations, are well known to those of ordinary skill in the art of medicine.

For local administration of viral vectors, previous clinical studies have demonstrated that up to 10¹³ pfu of virus can be injected with minimal toxicity. In human patients, 1×10⁹-1×10¹³ pfu are routinely used. See Habib et al. (1999) Human Gene Therapy 12:2019-2034. However, due to the ability of the delivery formulations disclosed herein to sequester viral vectors within a target tissues, it may be possible to administer even greater amounts of virus vector. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose is also modified to maximize anti-tumor and/or antiangiogenic activity.

### III. METHODS FOR REDUCING LEAKAGE OF DELIVERY FORMULATIONS

The presently disclosed subject matter provides methods of using the delivery formulations disclosed herein to reduce leakage of the delivery formulation from a target tissue after administration of the delivery formulation. The leakage from the target tissue can be a result of leakage from damaged blood vessels within the target tissue.

The term “damage”, and grammatical variants thereof, can refer to physical damage to a blood vessel as a result of the intrusion of an instrument used to administer a delivery formulation. For example, injection of vectors into tumors can result in damage to blood vessels normally found in the vicinity of the injection site. As used herein, “damaged” also refers to blood vessels that are abnormal even before the vectors are administered. For example, tumor blood vessels are frequently abnormal, and are often characterized by large pores in the vessel endothelium. Thus, leakage of a vector out of a target tissue and into the circulation and other healthy tissue (i.e., “non-target tissue”) can result from the damage to the blood vessels that is caused by the injection instrument (e.g., a needle), and can also result from the leakage of the vectors through pores that are present in damaged (i.e., abnormal) tumor blood vessels.

The delivery formulation, as disclosed herein, comprises a blocking agent having an initial low viscosity which rapidly increases to a higher viscosity after administration to a target tissue. The delivery formulation can further comprise in some embodiments a vector delivered to the target tissue, such as for example a viral or non-viral vector encoding a therapeutic polypeptide.

In some embodiments, the method comprises providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP and in some embodiments to at least about 1000 cP, whereby leakage of the delivery formulation into a damaged blood vessel either during or after administration of the delivery formulation is reduced.

In some embodiments, the vector is a viral vector and in some embodiments the viral vector is a retrovirus vector, a pseudotyped retrovirus vector, an adenovirus vector, an adeno-associated virus vector, a herpes virus vector, a vaccinia virus vector, a Semliki Forest virus vector, or a baculovirus vector. In some embodiments, the vector is a non-viral vector and in some embodiments the non-viral vector is selected from the group consisting of plasmids, bacteria, water-oil emulsions, polyethylene imines, dendrimers, micelles, microcapsules, liposomes, and cationic lipids.

In some embodiments, the polypeptide is a therapeutic polypeptide. In some embodiments, the therapeutic polypeptide is selected from the group consisting of reporter molecules, immunostimulatory molecules (e.g., a cytokine), enzymes that can convert prodrugs to drugs, tumor suppressor gene products, tumor antigens, apoptosis mediators, toxins, and anti-angiogenic factors.

In some embodiments, the viscosity of the blocking agent is less than about 20 cP before administration, and increases to at least about 100 cP after administration. In some embodiments, the viscosity of the blocking agent is less than about 200 cP before administration, and increases to at least about 1,000 cP after administration. In some embodiments the viscosity of the blocking agent increases to about 200 cP to about 100,000 cP after administration, depending on the rate of injection. In some embodiments the viscosity of the blocking agent increases to about 4,000 cP to about 100,000 cP after administration, depending on the rate of injection. Further, in some embodiments, the viscosity of the blocking agent increases to at least about 100 cP (and in some embodiments to at least about 1000 cP) within about one minute, in some embodiments within about 30 seconds, in some embodiments within about 10 seconds, and in some embodiments within about 1 sec or less after injection.

In some embodiments, the blocking agent comprises a poloxamer-containing formulation, which in some embodiments is present in a concentration ranging from about 10% to about 40% by weight, in some embodiments from about 12% to about 30% by weight, and in some embodiments from about 15% to about 21% by weight. In some embodiments, the blocking agent comprises an alginate-containing formulation, which in some embodiments is present in a concentration ranging from about 0.1% to about 8% by weight, in some embodiments from about 2% to about 8%, and in some embodiments from about 4% to about 6% by weight.

In some embodiments, the increase in viscosity of the blocking agent does not result in a significant trapping of the vector in the delivery formulation or a significant reduction in the efficiency of the viral vector to infect the target tissue.

The term “significant”, and grammatical variants thereof, refers to an amount (e.g., of trapping of a viral vector in a delivery formulation or of a reduction in infection efficiency) that would render the method ineffective for its intended purpose. In some embodiments, to determine whether or not a relationship is “significant” or has “significance”, statistical manipulations of the data can be performed to calculate a probability, expressed as a “p-value”. Those p-values that fall below a user-defined cutoff point are regarded as significant. In some embodiments, a p-value less than or equal to 0.05, in some embodiments less than 0.01, in some embodiments less than 0.005, and in some embodiments less than 0.001, are regarded as significant. Alternatively, significance can be expressed as a percent difference between two conditions that differ in one or more parameters. Thus, a “significant” difference can be in some embodiments a 50% difference, in some embodiments a 25% difference, in some embodiments a 20% difference, in some embodiments a 15% difference, in some embodiments a 10% difference, in some embodiments a 5% difference, in some embodiments a 2% difference, in some embodiments a 1% difference, and in some embodiments less than a 1% difference.

Thus, for example, “significant trapping” refers to a degree of trapping of a viral vector in a delivery formulation as a result of the viscosity change of the delivery formulation that results in an unacceptably low fraction of the viral vector being available for infecting the target tissue. Similarly, a “significant reduction in the efficiency of the viral vector infecting a target tissue” refers to a reduction in infection efficiency as a result of the viscosity change of the delivery formulation that negates the increased retention of the viral vector in the target tissue that the delivery formulation provides. In these embodiments, trapping and efficiency can be related (e.g., they can be inversely proportional), and some degree of trapping can be acceptable provided that the infection efficiency that results is not unacceptably low.

### IV. METHODS FOR DECREASING SYSTEMIC TOXICITY OF DELIVERY FORMULATIONS

The presently disclosed subject matter further provides methods for decreasing systemic toxicity of a delivery formulation. In some embodiments, the methods comprise providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP, and in some embodiments to at least about 1,000 cP, whereby systemic toxicity of the delivery formulation is decreased.

“Systemic toxicity” as used herein refers to toxicity experienced by a subject when a vector or the therapeutic agent leaks from a site of administration in the subject and enters the circulation of the subject. Systemic toxicity can result in the development of an immune response to the vector, expression of the therapeutic polypeptide in tissues where such expression is undesirable (for example, the expression of a cytotoxic therapeutic polypeptide in a normal tissue), or any other occurrence that would not be wanted in the subject, including death.

In some embodiments, the vector is a viral vector and in some embodiments the viral vector is a retrovirus vector, a pseudotyped retrovirus vector, an adenovirus vector, an adeno-associated virus vector, a herpes virus vector, a vaccinia virus vector, a Semliki Forest virus vector, or a baculovirus vector. In some embodiments, the vector is a non-viral vector and in some embodiments the non-viral vector is selected from the group consisting of plasmids, bacteria, water-oil emulsions, polyethylene imines, dendrimers, micelles, microcapsules, liposomes, and cationic lipids.

In some embodiments, the polypeptide is a therapeutic polypeptide. In some embodiments, the therapeutic polypeptide is selected from the group consisting of reporter molecules, immunostimulatory molecules (e.g., a cytokine), tumor suppressor gene products, tumor antigens, apoptosis mediators, toxins, and anti-angiogenic factors.

In some embodiments, the viscosity of the blocking agent is less than about 20 cP before administration, and increases to at least about 100 cP after administration. In some embodiments, the viscosity of the blocking agent is less than about 200 cP before administration, and increases to at least about 1,000 cP after administration. In some embodiments the viscosity of the blocking agent increases to about 200 cP to about 100,000 cP after administration, depending on the rate of injection. In some embodiments the viscosity of the blocking agent increases to about 4,000 cP to about 100,000 cP after administration, depending on the rate of injection. Further, in some embodiments, the viscosity of the blocking agent increases to at least about 100 cP (and in some embodiments to at least about 1,000 cP) within about one minute, in some embodiments within about 30 seconds, in some embodiments within about 10 seconds, and in some embodiments within about 1 sec or less after injection.

In some embodiments, the blocking agent comprises a poloxamer-containing formulation, which in some embodiments is present in a concentration ranging from about 10% to about 40% by weight, in some embodiments from about 12% to about 30% by weight, and in some embodiments from about 15% to about 21% by weight. In some embodiments, the blocking agent comprises an alginate-containing formulation, which in some embodiments is present in a concentration ranging from about 0.1% to about 8% by weight, in some embodiments from about 2% to about 8%, and in some embodiments from about 4% to about 6% by weight.

In some embodiments, the increase in viscosity of the blocking agent does not result in a significant trapping of the viral vector in the delivery formulation or a significant reduction in the efficiency of the viral vector to infect the target tissue.

### V. METHODS FOR REDUCING IMMUNE SYSTEM DETECTION OF DELIVERY FORMULATIONS

The presently disclosed subject matter also provides methods for reducing immune system detection of a delivery formulation in a subject. In some embodiments, the methods comprise providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP, and in some embodiments to at least about 1000 cP, whereby immune system detection in the subject of the delivery formulation is decreased.

A decrease in detection of the subject's immune system refers to a decrease in an immune response by the subject's immune system to a component (e.g., antigen) of the delivery formulation, including components of the vector, polypeptides encoded by the vector, and the blocking agent. The term “immune response” is meant to refer to any response to an antigen or antigenic determinant by the immune system of a vertebrate subject. Exemplary immune responses include humoral immune responses (e.g. production of antigen-specific antibodies) and cell-mediated immune responses (e.g. lymphocyte proliferation), as defined herein below. Representative molecules that can elicit an immune response and that can be measured to determine if a decrease in immune response has been achieved include interleukins, for example interleukin 2 (IL2) and interleukin 12 (IL12). Proliferation and/or cellular changes (e.g., cell differentiation) to cell classes of the immune system can also be measured to determine changes in immune response.

The term “immune system” includes all the cells, tissues, systems, structures and processes, including non-specific and specific categories, which provide a defense against antigenic molecules, including potential pathogens, in a vertebrate subject. As is well known in the art, the non-specific immune system includes phagocytic cells such as neutrophils, monocytes, tissue macrophages, Kupffer cells, alveolar macrophages, dendritic cells and microglia. The specific immune system refers to the cells and other structures that impart specific immunity within a host. Included among these cells are the lymphocytes, particularly the B cell lymphocytes and the T cell lymphocytes. These cells also include natural killer (NK) cells and lymphokine activated killer (LAK) cells. Additionally, antibody-producing cells, like B lymphocytes, and the antibodies produced by the antibody-producing cells are also included within the term “immune system”.

The term “systemic immune response” is meant to refer to an immune response in the lymph node-, spleen-, or gut-associated lymphoid tissues wherein cells, such as B lymphocytes, of the immune system are developed. For example, a systemic immune response can comprise the production of serum IgGs, which can also be measured to determine if a decrease in immune response has been achieved. Further, systemic immune response refers to antigen-specific antibodies circulating in the blood stream and antigen-specific cells in lymphoid tissue in systemic compartments such as the spleen and lymph nodes.

The terms “humoral immunity” or “humoral immune response” are meant to refer to the form of acquired immunity in which antibody molecules are secreted in response to antigenic stimulation.

The terms “cell-mediated immunity” and “cell-mediated immune response” are meant to refer to the immunological defense provided by lymphocytes, such as that defense provided by T cell lymphocytes when they come into close proximity to their target cells. A cell-mediated immune response also comprises lymphocyte proliferation. When “lymphocyte proliferation” is measured, the ability of lymphocytes to proliferate in response to specific antigen is measured. Lymphocyte proliferation is meant to refer to B cell, T-helper cell, or CTL cell proliferation.

The term “CTL response” is meant to refer to the ability of an antigen-specific cell to lyse and kill a cell expressing the specific antigen. Standard, art-recognized CTL assays can be performed to measure CTL activity.

In some embodiments, the vector is a viral vector and in some embodiments the viral vector is a retrovirus vector, a pseudotyped retrovirus vector, an adenovirus vector, an adeno-associated virus vector, a herpes virus vector, a vaccinia virus vector, a Semliki Forest virus vector, or a baculovirus vector. In some embodiments, the vector is a non-viral vector and in some embodiments the non-viral vector is selected from the group consisting of plasmids, bacteria, water-oil emulsions, polyethylene imines, dendrimers, micelles, microcapsules, liposomes, and cationic lipids.

In some embodiments, the polypeptide is a therapeutic polypeptide. In some embodiments, the therapeutic polypeptide is selected from the group consisting of reporter molecules, immunostimulatory molecules (e.g., a cytokine), enzymes that can convert prodrugs to drugs, tumor suppressor gene products, tumor antigens, apoptosis mediators, toxins, and anti-angiogenic factors.

In some embodiments, the viscosity of the blocking agent is less than about 20 cP before administration, and increases to at least about 100 cP after administration. In some embodiments, the viscosity of the blocking agent is less than about 200 cP before administration, and increases to at least about 1,000 cP after administration. In some embodiments the viscosity of the blocking agent increases to about 200 cP to about 100,000 cP after administration, depending on the rate of injection. In some embodiments the viscosity of the blocking agent increases to about 4,000 cP to about 100,000 cP after administration, depending on the rate of injection. Further, in some embodiments, the viscosity of the blocking agent increases to at least about 100 cP (and in some embodiments to at least about 1,000 cP) within about one minute, in some embodiments within about 30 seconds, in some embodiments within about 10 seconds, and in some embodiments within about 1 sec or less after injection.

In some embodiments, the blocking agent comprises a poloxamer-containing formulation, which in some embodiments is present in a concentration ranging from about 10% to about 40% by weight, in some embodiments from about 12% to about 30% by weight, and in some embodiments from about 15% to about 21% by weight. In some embodiments, the blocking agent comprises an alginate-containing formulation, which in some embodiments is present in a concentration ranging from about 0.1% to about 8% by weight, in some embodiments from about 2% to about 8%, and in some embodiments from about 4% to about 6% by weight.

In some embodiments, the increase in viscosity of the blocking agent does not result in a significant trapping of the viral vector in the delivery formulation or a significant reduction in the efficiency of the viral vector to infect the target tissue.

### VI. METHODS FOR INCREASING EFFICIENCY OF VECTOR DELIVERY TO TARGET TISSUES

Besides the substantial benefits realized from reduced systemic dissemination of vectors of decreased systemic toxicity and immune response, the delivery formulations of the present subject matter further provide the advantage of increasing efficiency of vector delivery to a target tissue. For example, in the case of a viral vector, sequestration of the viral vector at the target site results in significantly increased virus copy number within and contact with cells of a target tissue, which in turn can result in increased transgene expression within the target tissue. Therefore, fewer copy numbers of a virus vector are needed to achieve the same therapeutic benefit. Alternatively, since the delivery formulations of the presently disclosed subject matter facilitate sequestration of the viral vectors within the target tissue, even greater copy numbers of virus vector can be administered than is otherwise safe since sequestration reduces systemic toxicity. Either way, the overall net effect is an enhanced efficacy/toxicity ratio when the delivery formulations of the present subject matter are utilized in viral gene therapy, as compared to other formulations known in the art.

As such, the presently disclosed subject matter provides methods of increasing efficiency of vector delivery to a target tissue. In some embodiments, the methods comprise providing a delivery formulation comprising a blocking agent and one or more vectors encoding one or more polypeptides, wherein the delivery formulation is in a liquid form; and administering the delivery formulation to a target tissue in a subject, wherein the administering results in the viscosity of the blocking agent increasing to at least about 100 cP, and in some embodiments to at least about 1,000 cP, wherein the increase in viscosity of the blocking agent reduces leakage of the vector from the target tissue to thereby increase the efficiency of vector delivery to the target tissue. In some embodiments, the increased efficiency of vector delivery results in an increase in the expression of the therapeutic polypeptide in the target tissue of at least about 2-fold, in some embodiments at least about 4-fold when compared to the expression of the therapeutic polypeptide in the target tissue when an equivalent dosage of the viral vector is administered in the absence of the blocking agent.

In some embodiments, the vector is a viral vector and in some embodiments the viral vector is a retrovirus vector, a pseudotyped retrovirus vector, an adenovirus vector, an adeno-associated virus vector, a herpes virus vector, a vaccinia virus vector, a Semliki Forest virus vector, or a baculovirus vector. In some embodiments, the vector is a non-viral vector and in some embodiments the non-viral vector is selected from the group consisting of plasmids, bacteria, water-oil emulsions, polyethylene imines, dendrimers, micelles, microcapsules, liposomes, and cationic lipids.

In some embodiments, the polypeptide is a therapeutic polypeptide. In some embodiments, the therapeutic polypeptide is selected from the group consisting of reporter molecules, immunostimulatory molecules (e.g., a cytokine), enzymes that can convert prodrugs to drugs, tumor suppressor gene products, tumor antigens, apoptosis mediators, toxins, and anti-angiogenic factors.

In some embodiments, the viscosity of the blocking agent is less than about 20 cP before administration; and increases to at least about 100 cP after administration. In some embodiments, the viscosity of the blocking agent is less than about 200 cP before administration, and increases to at least about 1,000 cP after administration. In some embodiments the viscosity of the blocking agent increases to about 200 cP to about 100,000 cP after administration, depending on the rate of injection. In some embodiments the viscosity of the blocking agent increases to about 4,000 cP to about 100,000 cP after administration, depending on the rate of injection. Further, in some embodiments, the viscosity of the blocking agent increases to at least about 100 cP (and in some embodiments to at least about 1,000 cP) within about one minute, in some embodiments within about 30 seconds, in some embodiments within about 10 seconds, and in some embodiments within about 1 sec or less after injection.

In some embodiments, the blocking agent comprises a poloxamer-containing formulation, which in some embodiments is present in a concentration ranging from about 10% to about 40% by weight, in some embodiments from about 12% to about 30% by weight, and in some embodiments from about 15% to about 21% by weight. In some embodiments, the blocking agent comprises an alginate-containing formulation, which in some embodiments is present in a concentration ranging from about 0.1% to about 8% by weight, in some embodiments from about 2% to about 8%, and in some embodiments from about 4% to about 6% by weight.

In some embodiments, the increase in viscosity of the blocking agent does not result in a significant trapping of the viral vector in the delivery formulation or a significant reduction in the efficiency of the viral vector to infect the target tissue.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Examples 1-4

Direct intratumoral injection or infusion with viral vectors encoding therapeutic polypeptides is one strategy for improving the efficacy/toxicity ratio in viral gene therapy. It has been widely used in cancer clinic trials. Researchers often assume that viral vectors and transgene expression are confined to solid tumors after the infusion, thereby causing minimal toxicity in normal tissues (Lu et al., 1999; Paielli et al., 2000). On the other hand, researchers in some studies have observed that transgene expression in normal tissues can be as strong as that in tumors (Bramson et al., 1997; Lohr et al., 2001; Nasu et al., 1999; Tjuvajev et al., 1999; Toloza et al., 1996; Wang et al., 2003), that adenoviral vectors are present in systemic circulation of patients in clinical trials (DeWeese et al., 2001; Nemunaitis et al., 2001), and that some animals die within a short period after intratumoral infusion (Tjuvajev et al., 1999; Toloza et al., 1996). These observations indicate that viral vectors can escape from tumors during and/or after intratumoral infusion.

Although virus dissemination has been observed in preclinical and clinical studies, its kinetics and pathways are still controversial. Therefore, the biodistributions of adenoviral vectors and transgene expression in the tumor, liver, blood, and lung have been characterized and results reported herein at different time points after the infusion was started. Systemic virus dissemination was observed to occur mainly within the first 10 minutes (min). The amount of viruses disseminated into the liver could be one order of magnitude higher than that retained in the tumor, and the mechanism of dissemination was the infusion-induced convective transport of viral vectors into leaky tumor microvessels.

### Materials and Methods for Examples 1-4

Adenoviral vectors. The Ad5-based recombinant system was used to produce the non-replicating adenoviruses: AdCMVEGFP and AdCMVLuc encoding enhanced green fluorescence protein (EGFP) and luciferase, respectively. The cDNA for EGFP or luciferase was inserted into the E1 region of the adenovirus and the transgene expression was driven by the cytomegalovirus (CMV) promoter. Adenoviruses were propagated in 293 cells (ATCC, Manassas, Va., U.S.A.), harvested at 48 hrs after infection, and purified by the method of cesium chloride gradient centrifugation according to a standard protocol (Graham & Prevec, 1995). The viral vectors were stored in 10% glycerol at −82° C.

Tumor cell lines. Three tumor cell lines (4T1, B16.F10, and IMR-32) were used in Examples 1-4. 4T1 is a murine mammary carcinoma; B16.F10 is a metastatic subline of B16 murine melanoma; IMR-32 is a human neuroblastoma. All tumor cells were cultured in DMEM supplemented with 10% newborn bovine serum (Hyclone, Logan, Utah, U.S.A.) and 1% penicillin/Streptomycin (Gibco/Life Technologies, Grand Island, N.Y., U.S.A.) at 37′C with 5% CO₂ and 95% air. For tumor implantation in mice, the cells were removed from culture flasks with 0.25% trypsin/EDTA and rinsed twice in PBS.

Animals. Four to six week old female Balb/c and C57BL/6 mice were ordered from Charles River Laboratory (Wilmington, Mass., U.S.A.) for the implantation of 4T1 and B16.F10 tumors, respectively. Female Balb/c nude mice at the same age as Balb/c mice were ordered from the U.S. National Cancer Institute (Bethesda, Md., U.S.) for the implantation of IMR-32 tumor. For tumor cell implantation, mice were first anesthetized with an intraperitoneal injection (0.15 ml) of a mixture of ketamine and xylazine (80 mg ketamine and 10 mg xylazine per kg body weight). Then, one million tumor cells in 50 μl phosphate buffered saline (PBS) were injected subcutaneously into the right hind limb of mice. After implantation, the mice were placed on a warm pad until they woke up. All subcutaneous tumors were ready for experiments when they reached 5-8 mm in diameter. After experiments, fully anaesthetized mice were sacrificed with cervical dislocation. The use of animals and all experimental procedures involving animals in this study had been approved by the Duke University Institutional Animal Care & Use Committee, Durham, N.C., U.S.A. These procedures were also consistent with the United Kingdom Co-ordinating Committee on Cancer Research (UKCCCR) Guidelines (UKCCCR, 1998).

Intratumoral infusion. Mice were anesthetized with an intraperitoneal injection (0.15 ml) of a mixture of ketamine and xylazine (80 mg ketamine and 10 mg xylazine per kg body weight). The tip of a 30-gauge needle was carefully placed near the center of tumors by controlling the depth of needle insertion relative to the tumor size measured by a caliper. Virus suspension was infused into tumors via the needle mounted on a syringe pump (model 22, Harvard Apparatus Co., Cambridge, Mass., U.S.A.). The volume of infusion was fixed at 50 although the dose of viruses varied with experiments as indicated below. The infusion rate was chosen to be 1 μl/sec based on a preliminary study in which 50 μl of a solution of Evans blue dye or a suspension of an adenoviral vector for LacZ, AdCMVLacZ, was infused into 4T1 tumors, and in which it was observed that the distribution volume of Evans blue or β-galactosidase in tumors was the highest when the infusion rate was 1 μl/sec.

Luciferase and EGFP expression in different organs/tissues. At 24 or 48 hrs after intratumoral infusion of AdCMVLuc or AdCMVEGFP (2.0×10⁸ plaque forming units (pfu)), mice were anesthetized again with the same method as described above and transgene expression in tumors was examined. For luciferase analysis, 50 μl of aqueous D-luciferin solution was injected intraperitoneally into the mice placed on a warm pad at 20 min before the examination of bioluminescence in the body. The bioluminescence was recorded by the IN VIVO IMAGING SYSTEM™ (IVIS) (Xenogen Corp., Alameda, Calif., U.S.A.), with an exposure time of 30 sec (Wang et al., 2003) and the bioluminescence intensity was represented by pseudo-colors as indicated by a color bar. The final images were generated by superimposing the pseudo-color bioluminescence images on the reference images. For EGFP analysis, tissue samples from the blood, lung, liver, and tumor were harvested. Except the blood, tissue samples were sectioned into thin slices (300 μm thick), using a VIBRATOME® (Model 3000; Technical Products International, St. Louis, Mo., U.S.A.). EGFP expression in the blood and sliced tissues was examined under a confocal laser-scanning microscope (LSM 510, Carl Zeiss, Hanover, Md., U.S.A.).

In a separate experiment, 10⁶ luciferase-expressing 4T1 cells were injected in a 100-μl suspension into the tail vein of Balb/c mice anesthetized with the same method as described herein. At both 10 min and 24 hrs after the cell injection, luciferase expression in the body was examined using the same method described herein.

Qualitative analysis of adenoviruses in the blood. At predetermined time points (25 s, 50 s, 5 min, 10 min, 2 hr, and 24 hr) after starting the infusion of AdCMVEGFP (3.0×10⁸ pfu) into 4T1 tumors, blood samples (approximately 20 μl in each sample) from anesthetized mice were collected through the orbital sinus with a heparinized capillary, stored at room temperature for 10 minutes, and centrifuged at 10,000×g for 3 min to separate the plasma from blood cells. The plasma and cell suspension were then exposed to four cycles of thawing (37° C.) and freezing (−196° C. in liquid nitrogen) treatment. 1 μl of plasma solution or suspension of lysed blood cells was added into 96-well plates with 70-80% confluent 293 cells. 24 hrs later, EGFP expression in these cells was examined under a fluorescence microscope (AXIOVER™ 100®, Carl Zeiss).

Quantitative analysis of adenovirus dissemination in liver and tumors. At 10 min after the infusion of AdCMVEGFP (3.0×10⁸ pfu) into 4T1 tumors, mice were sacrificed with cervical dislocation and the liver and tumors were immediately harvested, frozen in liquid nitrogen, and stored at −82° C. For viral DNA analysis, the tissue samples were immersed in liquid nitrogen and ground into fine tissue powder in a mortar. Viral DNAs was isolated from the tissue powder using a DNEASY® Tissue kit (Qiagen, Clarita, Calif., U.S.A.). The kit mini column was eluted twice with 200 μl of Tris buffer (10 mM, pH=8.5) to obtain viral DNA solution, in which Ad E4 copy number was measured with the SMART CYCLER™ System (Cepheid, Sunnyvale, Calif., U.S.A.). In the real-time PCR analysis, the primers and probes were purchased from Sigma-Genosis (Woodlands, Tex., U.S.A.). The forward primer sequence for Ad E4 region was: 5′-TGACACGCATACTCGGAGCTA-3′: 34885-34905 (SEQ ID NO:1); the reverse primer sequence was: 5′-TTTGAGCAGCACCTTGCATT-3′: 34977-34958 (SEQ ID NO:2); the fluorogenic probe sequence was:

[DFAM]CGCCGCCCATGCAACAAGCTT[DBH1]: 34930-34951 (SEQ ID NO:3) (Adachi et al., 2001). Each PCR reaction was performed in duplicate in a 25-μl reaction mixture containing 0.15 μl of AMPLITAQ GOLD® DNA polymerase (5 U/μl) (Applied Biosystems, Foster City, Calif., U.S.A.), 300 nM each primer, and 200 nM fluorogenic probe. The amplification included an initial heating at 95° C. for 10 min, followed by 40 cycles at 95° C. for 15 sec and 60° C. for 45 sec. The standard curve was generated by using serial dilutions of pFG140 plasmid DNA (Microbix Biosystems Inc., Ontario, Canada) in the genomic liver DNA solution.

### Example 1

### Systemic AdCMVLuc Dissemination from Tumors

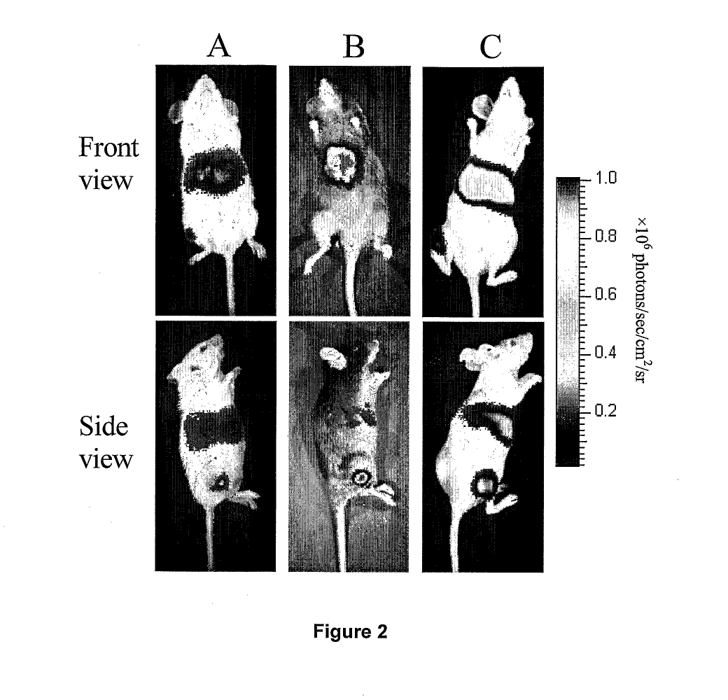

To demonstrate the systemic dissemination of viral vectors, AdCMVLuc suspension was infused into two murine tumor models, 4T1 and B16.F10, respectively, and one human tumor xenograft, IMR-32. Bleeding was observed in many tumors immediately after the removal of the infusion needle, but the amount of bleeding was much less than the infused volume (i.e., 50 μl). At 24 hours (hrs) after infusion, strong bioluminescence, an indication of luciferase expression, was observed in the liver and tumor in all animal models (see FIGS. 2A-2C). The bioluminescence from the liver indicated that AdCMVLuc had escaped from tumors in local gene delivery, because luciferase is an exogenous and non-secretable protein. It is normally retained in transfected, viable cells.

### Example 2

### Kinetics of Adenovirus Dissemination

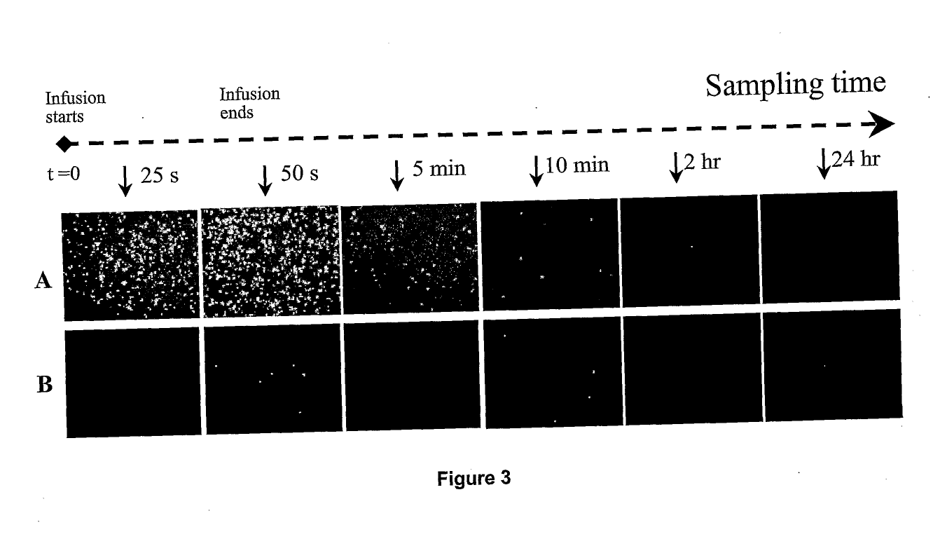

The plasma from blood samples at different time points, both during and after the intratumoral infusion of AdCMVEGFP, contained the viral vectors that could transfect 293 cells in vitro (see FIG. 3A). Qualitatively, the amount of AdCMVEGFP in the plasma reached the peak at the end of intratumoral infusion and then decreased quickly with time. It was significantly lower than the peak value at 10 min and nearly undetectable at 2 hrs after the infusion. Cells from blood samples contained only a few AdCMVEGFP that could transfect 293 cells in vitro (see FIG. 3B), indicating that the virus dissemination was not mediated by transfected tumor or blood-borne cells. This is believed to be the first direct evidence demonstrating adenovirus dissemination during intratumoral infusion.

### Example 3

### Role of Tumor or Blood-Borne Cells in Virus Dissemination

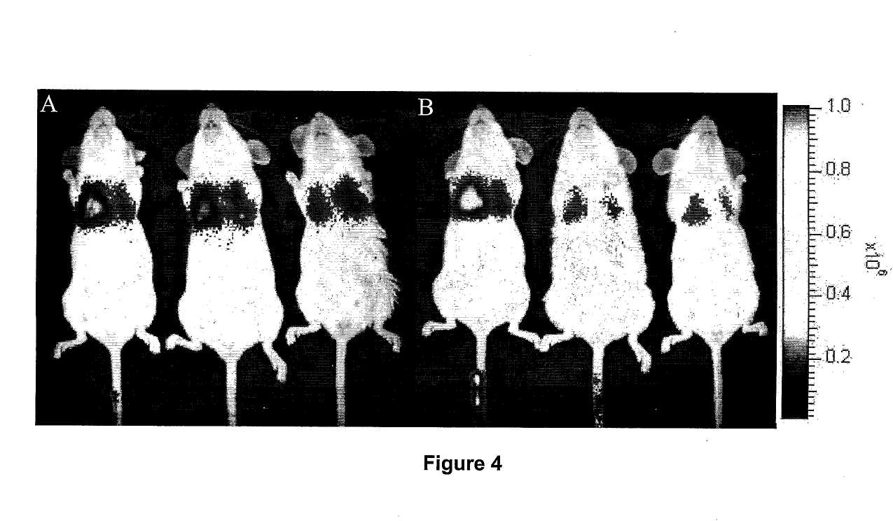

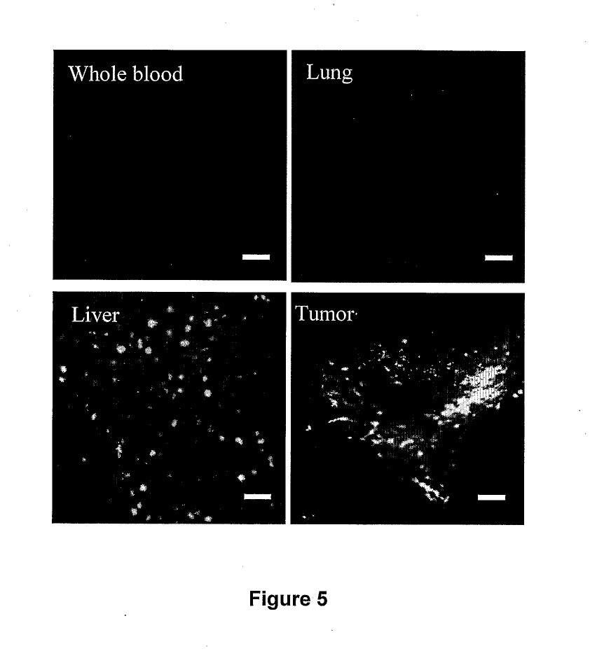

To further investigate the role of tumor or blood-borne cells in virus dissemination, two additional experiments were performed. Firstly, luciferase-expressing cells were infused into the tail vein of mice, and it was observed that nearly all infused cells accumulated in the lung (FIGS. 4A and 4B). Secondly, AdCMVEGFP vectors were infused into 4T1 tumors and EGFP expression examined in the liver, tumor, lung, and blood at 24 hrs after the infusion. EGFP expression was observed in liver and tumor tissues but EGFP-expressing cells could not be detected in the lung and blood (FIG. 5). These results further confirmed that virus dissemination was not mediated by transfected tumor or blood-borne cells.

### Example 4

### Quantitative Measurement of Adenoviruses in Liver and Tumor Tissues

To quantitatively understand the significance of systemic adenovirus dissemination at early time points, the copy numbers of adenoviruses were quantified in liver and tumor tissues at 10 min after intratumoral infusion of AdCMVEGFP. This time point was chosen for three reasons. Firstly, data in the literature show that adenoviruses have a plasma half-life of ˜2 min (Alemany et al., 2000; Worgall et al., 1997) (see also FIG. 3A). Thus, approximately 97% of disseminated viruses have been cleared from the blood at 10 min. Secondly, the clearance in mice is mainly through the liver (Hackett et al., 2000; Herz & Gerard, 1993). Finally, the amount of active adenoviral vectors in the liver decrease with time (Worgall et al., 1997). As a result, the copy numbers of adenoviruses in the liver at 10 min after the infusion provided the best estimate of the total amount of adenoviruses disseminated from the tumor.

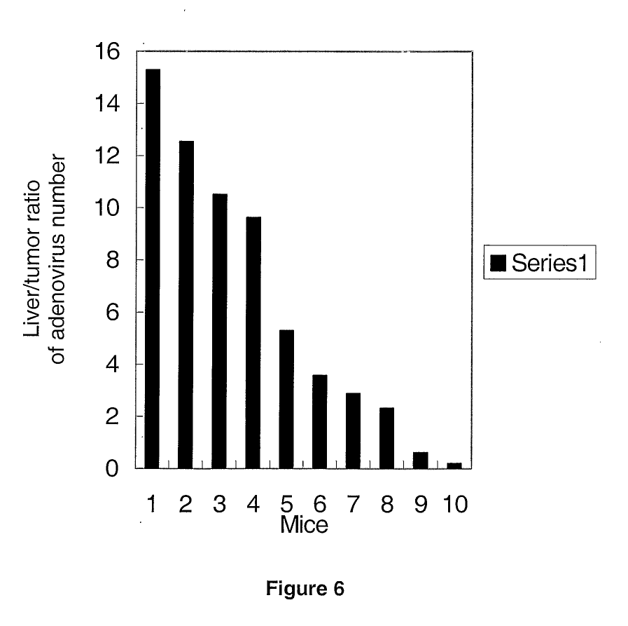

The ratio of the copy numbers of adenoviruses between liver and tumor is shown in FIG. 6. The minimal value was 0.2; 8 out of 10 mice had a ratio greater than 2; and 4 mice had a ratio close to or greater than 10. These data demonstrated that a significant amount of adenoviruses had disseminated from tumors within 10 min after the intratumoral infusion.

### Discussion of Examples 1-4

Systemic virus dissemination was observed in all three animal models after intratumoral infusion of adenoviral vectors. In 4T1 tumors, the dissemination occurred mainly during the first 10 min after the infusion was started. The amount of viruses disseminated into the liver could be one order of magnitude higher than that retained in the tumor. The blood and lung in mice contained few transfected cells after the intratumoral infusion. However, tumor cells injected intravenously into the mice accumulated mainly in the lung.

Potentially, the mechanisms of dissemination include cell-mediated transport and diffusion and convection of viral vectors from the interstitial space into the lumen of tumor microvessels. It has been previously shown that the diffusion process is too slow to cause the virus dissemination after intratumoral infusion (Wang et al., 2003). Based on diffusion, the maximum amount of disseminated viruses would be several orders of magnitude less than that retained in the tumor. This was inconsistent with the observations shown in FIGS. 2, 3; 5 and 6. Therefore, diffusion is negligible during the virus dissemination.

Cell-mediated transport was likely to be insignificant as well. The reasons are as follows. First, the amount of adenoviral vectors associated with cells in blood samples shown in FIG. 3B was extremely low compared with that in the plasma. Even the cell-associated vectors could be originally in the plasma and then non-specifically bind to erythrocytes (Cichon et al., 2003) or be co-precipitated with blood cells during the centrifuging process. Second, most tumor cells injected intravenously accumulated in the lung (FIGS. 4A and 4B), which was consistent with data in the literature that lung is the primary target of metastatic tumor cells in mice (Lee, 1983). Third, few transfected cells existed in the blood and lung at 24 hrs after the intratumoral infusion (FIG. 5). Therefore, the observations shown in FIG. 3B indicated that blood-borne cells played an insignificant role in the virus dissemination, while the observations shown in FIGS. 4A-4B and FIG. 5 indicated that the virus dissemination was not mediated by transfected tumor cells either.

Convection is in general negligible in the center of solid tumors, due to uniformly elevated interstitial fluid pressure and intrinsic leakiness of tumor microvessels (Jain, 1997). Convection is important only during the intratumoral infusion and the first few minutes after the intratumoral infusion when a fluid pressure gradient is created around the tip of the infusion needle. After the infusion, the pressure gradient should decrease rapidly with time since (a) the equilibrium time constant between the microvascular pressure and the interstitial fluid pressure is approximately 10 seconds (sec) (Netti et al., 1995) and (b) the microvascular pressure in the tumor should return to the un-disturbed level within a few minutes after the infusion, due to blood circulation. Therefore, convection can only cause virus dissemination at early time points but becomes insignificant at late time points.

Taken together, the data of Examples 1-4 indicates virus dissemination occurred after the adenoviral vectors entered the system circulation via infusion-induced convective transport. The entrances were the leaky microvessels that were either intrinsically hyperpermeable, with pores in the microvessel wall being larger than adenoviruses (Hobbs et al., 1998; Yuan, 1998) or damaged by the infusion procedures. In the blood, adenoviruses were rapidly taken up by Kupffer cells and hepatocytes in the liver, with a plasma half-life of ˜2 min (Alemany et al., 2000). The dissemination of the adenoviral vectors was significant only during the first few minutes (<10 min) after the intratumoral infusion was started.

The ratio of copy numbers of adenoviruses between liver and tumor shown in FIG. 6 had a large variation among different animals. This result was qualitatively consistent with the ratio of bioluminescence intensity between liver and tumor after intratumoral infusion of AdCMVLuc. Such variations were unlikely to be caused by the experimental procedures because the tip of the needle had been carefully placed near the center of all tumors and the infusion protocol had been standardized by using a syringe pump. The variation could be due to the heterogeneous distribution of microvessels, a hallmark of solid tumors. As *a result, the number of leaky microvessels involved in virus dissemination would depend on vascularization and location of the needle tip in tumors.

Systemic virus dissemination after intratumoral infusion has been investigated in previous studies (DeWeese et al., 2001; Nemunaitis et al., 2001; Sauthoff et al., 2003). Sauthoff et al have detected a self-replicating adenoviral vector in mouse blood at 10 min after intratumoral infusion, based on the assay of virus titration on 293 cells (Sauthoff et al., 2003). The number of viral DNA copies in the blood is one order of magnitude lower than that retained in the tumor and no adenoviruses can be detected in the liver during the first week after the intratumoral infusion. The liver data are inconsistent with those in the literature, which have demonstrated that nearly 99% of adenoviruses in the systemic circulation will eventually accumulate in the liver of mice (Hackett et al., 2000; Herz & Gerard, 1993). This discrepancy is likely to be caused by the inappropriate use of virus titration assay. It is known that most adenoviruses in the liver are internalized by Kupffer cells or hepatocytes. The internalization and the subsequent intracellular trafficking of adenoviruses might make these vectors lose their ability to transfect 293 cells. Therefore, a better way to determine the virus accumulation in the liver is to quantify the copy number of unique viral DNA sequences as shown in the present Examples. Sauthoff et al. have also shown that the adenoviral vectors can be detected in the blood at 2 to 8 weeks after the intratumoral infusion, presumably due to the replication of vectors in tumors (Sauthoff et al., 2003). Non-replicating vectors were used in Examples 1-4 so that virus dissemination at late time points due to viral replication was not observed.

In a clinical study, the amount of adenoviral DNA in the blood at 30 min after the intratumoral infusion has varied between 0.007% and 0.129% of that infused into tumors (DeWeese et al., 2001). The amount of adenoviruses disseminated into the liver or other normal tissues is not determined in patients. Based on the blood data alone, it is difficult to estimate the amount of disseminated adenoviruses because it is likely that most adenoviruses disseminated during the intratumoral infusion have already been taken up by the liver at 30 min (Alemany et al., 2000) (see also FIGS. 3 and 6).

In summary, the data presented in Examples 1-4 demonstrates that the virus dissemination occurred during the first few minutes after the intratumoral infusion was started. Dissemination was most likely due to infusion-induced convective transport of viral vectors into leaky tumor microvessels. These findings support the methods and materials disclosed herein, which provide delivery systems that can increase the concentration of vectors within solid tumors and simultaneously reduce the amount of disseminated vectors in normal tissues, particularly in the liver, thereby improving the efficacy/toxicity ratio in cancer gene therapy.

### Example 5

Examples 1-4 provide data indicating systemic virus vector dissemination resulting from infusion and/or injection of the virus vector into a target tissue for gene therapy can result at least in part due to convection activity. To directly demonstrate the effects of convection on virus dissemination, the dependence of adenovirus dissemination on infusion rate, volume, and dose was investigated and results disclosed in the present Example. Further, transgene expressions in tumor and liver tissues were quantified under infusion conditions.

The data indicate that virus dissemination was determined mainly by the infusion dose and that the amount of transgene expression in the tumor depended on the distribution volume of viral vectors in the tumor as well as the infusion dose.

### Materials and Methods for Example 5

Tumor model. A mouse mammary carcinoma cell line (4T1) was used as the tumor model. One million 4T1 cells in 50 μl PBS were subcutaneously injected into the right hind leg of four to six week old syngeneic female Balb/c mice (Charles River Laboratory, Wilmington, Mass., U.S.A.) after the animals were anesthetized with i.p. injection of a cocktail of ketamine and xylazine (80 mg ketamine and 10 mg xylazine per kg body weight). The subcutaneous tumors were used in experiments when they reached 5 to 8 mm in diameter. To minimize effects of tumor size on experimental data, tumors were randomly separated into different experimental groups. The animal protocol was approved by the Duke University Institutional Animal Care & Use Committee (Duke University, Durham, N.C., U.S.A.).

Adenoviral vector. An Ad5-based recombinant system was used to produce an adenoviral vector, AdCMVLuc, encoding luciferase. Adenoviruses were propagated in 293 cells (ATCC, Manassas, Va., U.S.A.), harvested at 48 hrs after infection, and purified by cesium chloride gradient centrifugation according to a standard protocol (Graham & Prevec, 1991).

Quantification of luciferase expression. AdCMVLuc suspension was infused into 4T1 tumors after mice were anesthetized using the protocol described herein. The infusion was performed via a 30-gauge needle mounted on a syringe pump (model 22, Harvard Apparatus Co., Cambridge, Mass., U.S.A.) as disclosed in detail in Examples 1-4. Luciferase expressions in the liver and tumor were quantified at different time points after intratumoral infusion of AdCMVLuc, using the Xenogen IVIS (Alameda, Calif., U.S.A.) as disclosed in detail in Examples 1-4.

Distribution of Evans blue-labeled albumin. Evans blue-labeled albumin solution was prepared by mixing 0.04% Evans blue and 0.1% albumin in 0.9% saline. The solution was infused into 4T1 tumors, following the same procedure as that for viral vector infusion described herein. After the infusion, the mice were immediately sacrificed by cervical dislocation. The tumors were harvested, mounted on a specimen block, transferred to the stage of the VIBRATOME® tissue sectioning system (model 3000, Technical Products International, St. Louis, Mo., U.S.A.), and sectioned into 300 μm slices. The slices were mounted on glass slides and scanned into a computer with an OPTIC PRO™ document scanner, Model 12000P (Plustek, Taipei City, Taiwan, China).

Statistical analysis. The Mann-Whitney U test was used to compare the difference between two unpaired groups. The difference was considered to be significant if the P-value was less than 0.05.

### Results and Discussion for Example 5

Data in the literature have demonstrated that more than 95% of adenoviruses in mouse systemic circulation will eventually accumulate in the liver (Wang & Yuan, 2006). As a result, adenoviral vectors accumulate mainly in the liver after they disseminate from the tumor after intratumoral infusion and the amount of luciferase, a non-secretable transgene product, in the liver correlates with the number of disseminated adenoviruses (see Examples 1-4). Luciferase expression in other tissues was negligible and thus not examined.

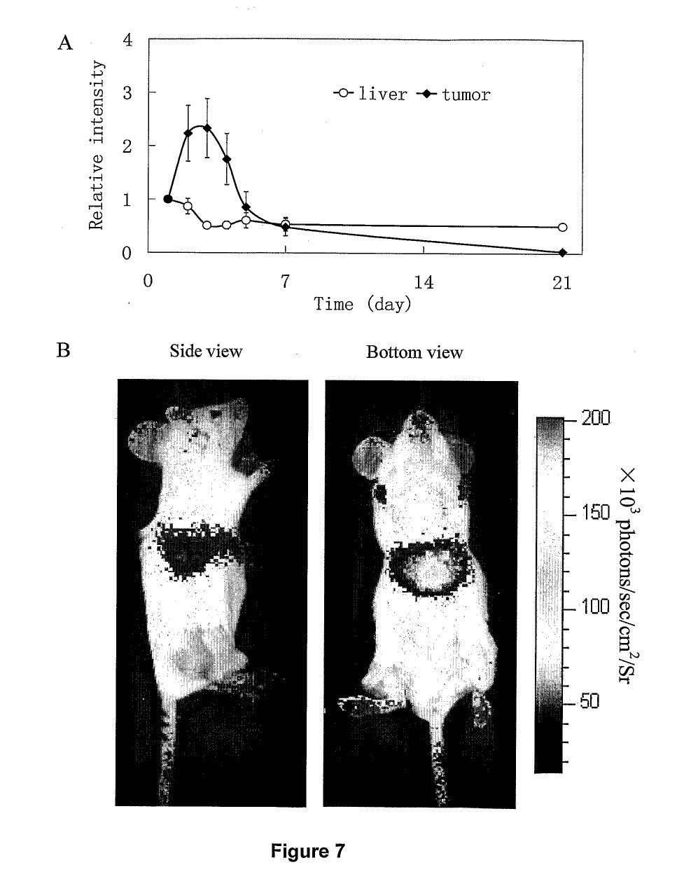

Transgene expression in the liver and targeted tumor is generally time-dependent. Luciferase expression in the liver decreased initially and then reached a plateau at three days after intratumoral infusion (FIG. 7A). There was no significant change in the luciferase expression in the liver between 3 and 21 days (P>0.05). Luciferase expression in the tumor increased during the first two to three days and then decreased exponentially (FIG. 7A). It could not be detected at 21 days after the infusion. Further, at 21 days, bioluminescence was observed in the liver, ears, nose, feet, and tail (FIG. 7B), which could not be detected during the first seven days after the intratumoral infusion. Sources of the bioluminescence in these tissues has not been determined.

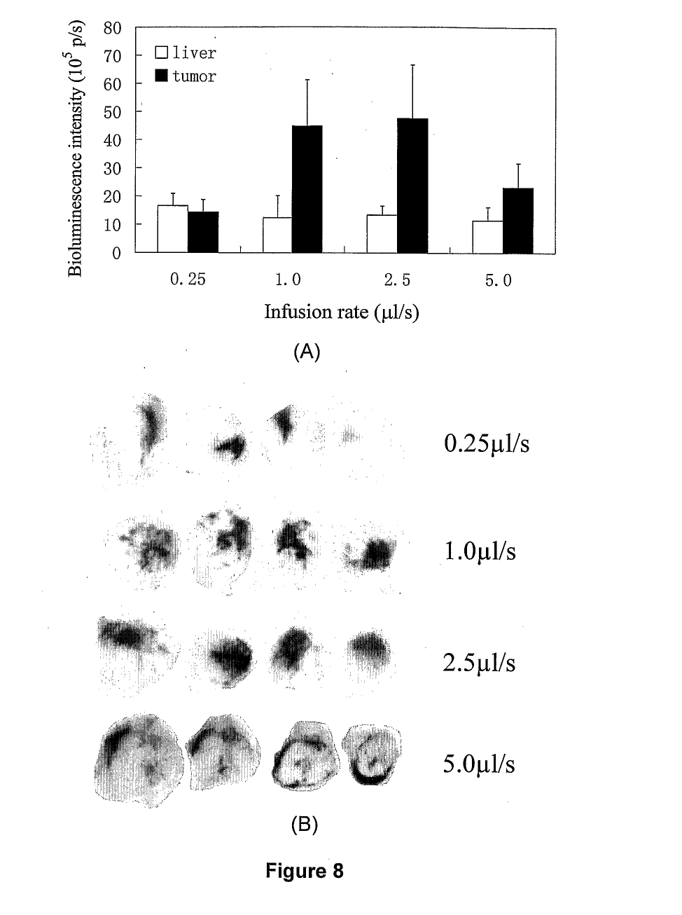

Based on the data shown in FIG. 7A, it was decided to quantify luciferase expression only at two days after intratumoral infusion in the following three experiments. First, effects of infusion rate on luciferase expression were investigated. In this experiment, adenoviral vectors were infused into the tumor at a rate between 0.25 and 5.0 μl/sec. This range of infusion rate covered the values used in previous studies. The infusion dose and volume per tumor were fixed at 2.0×10⁸ pfu and 50 μl, respectively. As a result, the time period of infusion varied, according to the infusion rate. Bioluminescence intensity observed in the liver was independent of the infusion rate at two days after intratumoral infusion (FIG. 8A), indicating that the infusion rate had minimal effects on virus dissemination. The mean bioluminescence intensity was a bell-shaped function of the infusion rate (FIG. 8A) but the difference between any pairs of data in tumor or liver was not statistically significant (P>0.05). The lack of significance was presumably due to the heterogeneity in tumor tissues which has also been observed in previous studies (McGuire & Yuan, 2001).

To understand why the intensity was low at the infusion rate of 5.0 μl/sec, the Evans blue-labeled albumin was infused into tumors at different rates and its distribution examined in tumors after the infusion. The results are shown in FIG. 8B. It was observed that albumin concentration was high near the needle track and low far away from the track in all experimental groups. It was also observed that the Evans blue-labeled albumin accumulated only in the periphery of tumors if the infusion rate was maintained at 5.0 μl/sec, indicating that albumin leaked out through some cracks in these tumors. Crack formation has been reported in previous studies of intratumoral infusion (McGuire & Yuan, 2001; Dillehay, 1997; Boucher et al., 1998). It is likely due to the presence of necrotic regions and blood pools as well as abnormal assembly of extracellular matrix in tumors. At both macroscopic (i.e., necrotic regions and blood pools) and microscopic (i.e., abnormal assembly of extracellular matrix) levels, these defects form weak structures can be ruptured to form macroscopic cracks during the infusion. Qualitatively, the images shown in FIG. 8B also demonstrated that the pattern of albumin distribution correlated with the bioluminescence intensity in the tumor shown in FIG. 8A. Although infusion-induced convection of albumin differed quantitatively from that of viral vectors, the results shown in FIG. 8 suggested that transgene expression depended on the distribution volume of adenoviral vectors in the tumor.

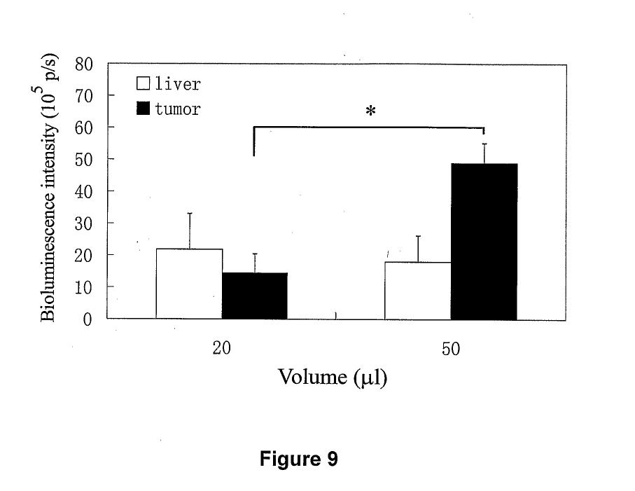

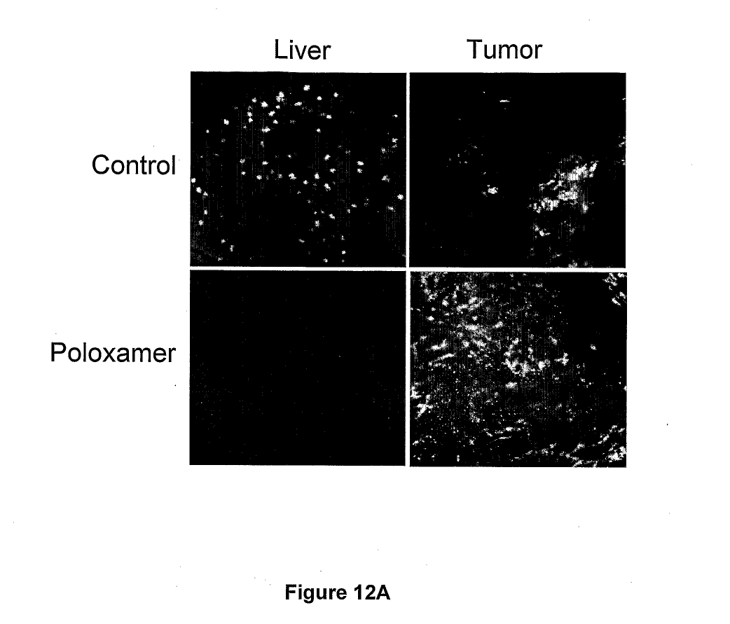

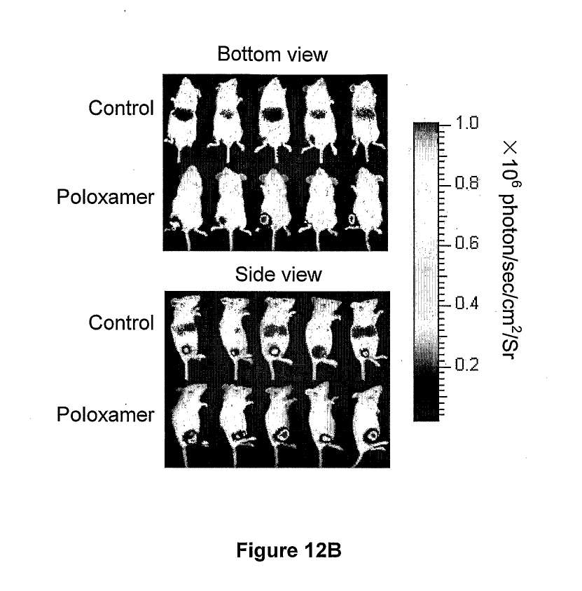

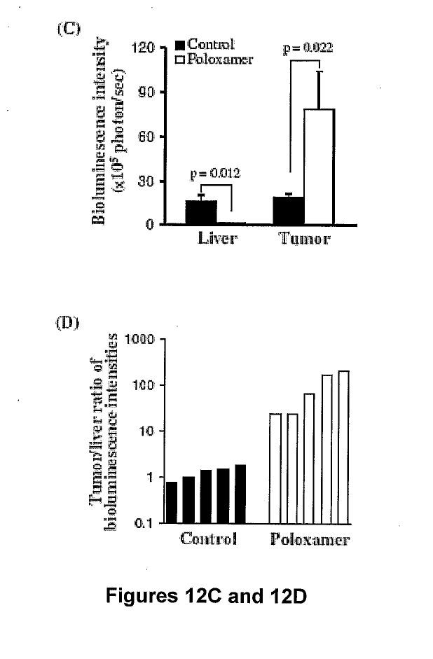

Second, the infusion dose and rate were fixed at 2.0×10⁸ pfu/tumor and 1.0 μl/sec, respectively, and varied the infusion volume from 20 to 50 μl. As a result, the time period of infusion and the virus concentration in the infusate were varied, according to the infusion volume. Again, it was observed that the bioluminescence intensity in the liver was independent of the infusion volume (FIG. 9), indicating that the infusion volume had minimal effects on the virus dissemination. Luciferase expression in tumors increased with increasing infusion volume (P<0.05) (FIG. 12). Also, different volumes (i.e., 20 and 50 μl) of the Evans blue-labeled albumin solution were infused into tumors. The distribution volume of Evans blue-labeled albumin was consistent with the luciferase expression in tumors. These results demonstrate that transgene expression in the tumor was affected by the distribution volume of adenoviral vectors.

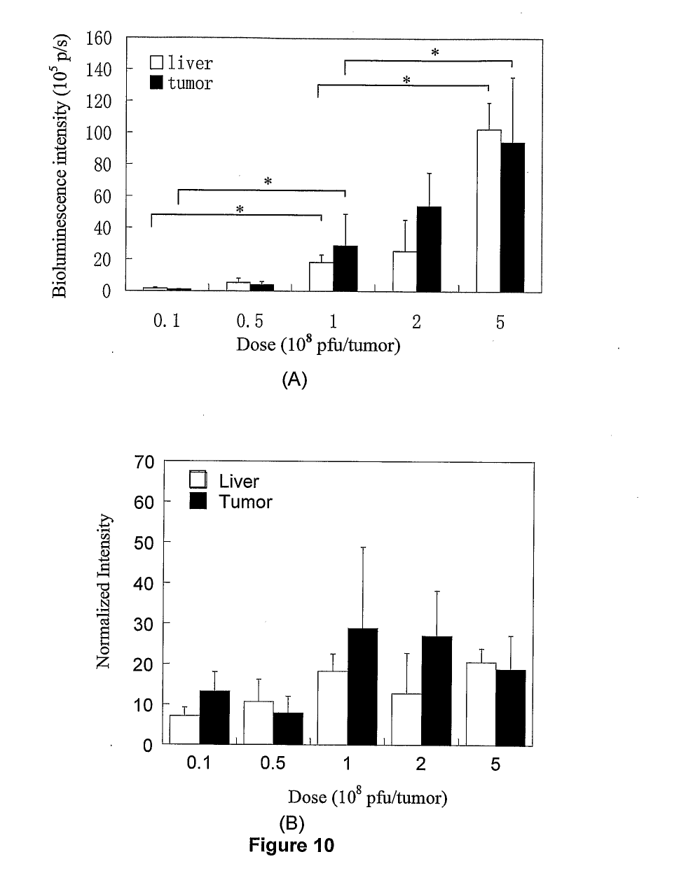

Finally, the dose of viral vectors was varied from 0.1×10⁸ to 5.0×10⁸ pfu/tumor by changing the virus concentration in the infusate while fixing the infusion rate and volume at 1.0 μl/sec and 50 μl, respectively. It was observed that the bioluminescence intensity in both tumor and liver tissues increased with increasing the dose of infusion (FIG. 10). The mechanism of the dose-dependence is likely to be that transgene expression is a sigmoidal function of the local concentration of viral vectors (Bristol et al., 2000), which in turn depends on the dose of infusion. However, the bioluminescence intensities normalized by the dose of infusion in both tissues were independent of the dose (FIG. 10B), except that the normalized intensity in the liver at the dose of 5.0×10⁸ pfu/tumor was significantly higher than the value of the same variable at the dose of 0.1×10⁸ pfu/tumor (P=0.03). Furthermore, it was observed that four out of nine mice in the dose group of 5.0×10⁸ pfu/tumor died within 30 min after intratumoral infusion, whereas no mice died in other dose groups.

In summary, the present Example demonstrates that virus dissemination can be determined by the dose of intratumoral infusion, whereas transgene expression in the tumor depended on not only the infusion dose but also the infusion volume. These data provided further evidence to support the conclusion drawn in Examples 1-4 that virus dissemination can be caused by infusion-induced convective transport in tumors.

### Example 6

Although intratumoral injection and/or infusion is a promising approach for gene therapy treatments of tumors, potential problems exist related to systemic dissemination from treated tumors to normal tissues during and after the infusion. To address this problem, a new delivery method for reducing the virus dissemination was developed as disclosed herein. In the embodiments disclosed in this example, the following rationale was pursued. Virus dissemination is due to convective transport of viral vectors into the systemic circulation (see Examples 1-5) and the rate of convection is inversely proportional to the viscosity of fluids. Therefore, the virus dissemination will be reduced if the viscosity of virus suspension is increased. On the other hand, merely increasing the viscosity can make the infusion too difficult to be performed in the clinic. To avoid this problem and yet increase the viscosity of virus suspension in tumor tissues, a poloxamer, that is, an ABA block polymer including poly(ethylene oxide) and poly(propylene oxide) units (Hecht et al. 1995; Wang & Johnston, 1991), was used as a delivery vehicle in the embodiments disclosed in this Example. Poloxamer is biocompatible. Its viscosity can increase two to three orders of magnitude when the temperature is increased from 4° C. to 37° C.

To test the feasibility of the method, a poloxamer 407 solution was prepared at 4° C., mixed with a suspension of adenoviral vectors, and the mixture infused into tumors. It was observed that the poloxamer solution could be injected easily, and after intratumoral infusion, the poloxamer-based blocking agent could reduce virus dissemination by two orders of magnitude and significantly increase transgene expression in solid tumors, as disclosed in detail in the present Example.

### Materials and Methods for Example 6

Tumor model. A mouse mammary carcinoma cell line (4T1) was used as the tumor model (Wang et al., 2003). One million 4T1 cells in 50 μl PBS were subcutaneously injected into the right hind leg of four to six week old syngeneic female Balb/c mice (Charles River Laboratory, Wilmington, Mass., U.S.A.) after the animals were anesthetized with i.p. injection of a cocktail of ketamine and xylazine (80 mg ketamine and 10 mg xylazine per kg body weight). The subcutaneous tumors were used in experiments when they reached 5 to 8 mm in diameter. The animal protocol was approved by the Duke University Institutional Animal Care & Use Committee.

Adenoviral vectors. An Ad5-based recombinant system was used to produce the adenoviral vectors, AdCMVEGFP and AdCMVLuc, encoding enhanced green fluorescence protein (EGFP) and luciferase, respectively (Wang et al., 2003). All adenoviral vectors were propagated in 293 cells (ATCC, Manassas, Va., U.S.A.) and stored in 10% glycerol at −80° C.

Poloxamer solution. Poloxamer 407 was purchased from BASF Corporation (Mount Olive, N.J., U.S.A.). The poloxamer solution was prepared by dissolving poloxamer powder into Opti-med solution at 4° C. Brookfield DV-III Rheometer (Brookfield, Mass.) was used to measure the viscosity of solutions at different shear rates and temperatures. The measurement at each shear rate and temperature was repeated three times.

Intratumoral infusion. 50 μl of viral vector suspension was infused into 4T1 tumors at 1 μl/sec via a 30-gauge needle mounted on a Model 22 Harvard syringe pump (Harvard Apparatus Co., Cambridge, Mass., U.S.A.) as disclosed in detail in Examples 1-4. The dose of infusion was 3.0×10⁸ pfu/tumor for AdCMVEGFP and 2.0×10⁸ pfu/tumor for AdCMVLuc.

Analysis of adenoviruses in the blood. Blood samples were collected from anesthetized mice through the orbital sinus with a heparinized capillary immediately after the intratumoral infusion of AdCMVEGFP as disclosed in detail in Examples 1-4. They were centrifuged at 14,000 rpm for 3 min to separate the plasma from cells. Then, 1 μl of plasma was added into each well in 96-well plates with 70-80% confluent 293 cells. After 24 hrs, the number of EGFP positive cells, which could be used as a measure of the amount of adenoviruses in the plasma, was examined under a fluorescence microscope as disclosed in detail in Examples 1-4.

Quantification of adenovirus copy numbers in liver and tumor tissues. The details of the quantification procedures have been previously described in detail in Examples 1-4. In brief, mice were sacrificed at 10 min after the intratumoral infusion of AdCMVEGFP. The livers and tumors were harvested, frozen in liquid nitrogen, and ground into fine tissue powder in a mortar. Viral DNAs were isolated from the tissue powder using a DNEASY® Tissue kit (Qiagen, Clarita, Calif., U.S.A.) and its copy number was determined, using the real-time PCR technique.

Analysis of transgene expression after the intratumoral infusion. For the analysis of EGFP expression, samples of liver and tumor were harvested at 24 hrs after the infusion of AdCMVEGFP. The tissue samples were sectioned into 300-μm slices with a VIBRATOME® (Model 3000; Technical Products International, St. Louis, Mo., U.S.A.) and examined under a confocal laser-scanning microscope (LSM 510, Carl Zeiss, Thornwood, N.Y., U.S.A.). This experiment was repeated three times and the typical results are shown in the present Example. For the analysis of luciferase expression, bioluminescence images of mice were acquired at 24 hrs after the infusion of AdCMVLuc, using the IN VIVO IMAGING SYSTEM™ (IVIS) (Xenogen Corp., Alameda, Calif., U.S.A.).

Tail vein injection of nanosphere or AdCMVLuc suspension. Suspension of rhodamine-labeled polystyrene nanospheres (114±1.8 nm in diameter, Polysciences, Inc., Warrington, Pa., U.S.A.) or AdCMVLuc was mixed with either PBS or poloxamer solution and stored at 4° C. before use. In experiments, the tail vein of anesthetized Balb/c mice was cannulated with a 30G needle connected to a tubing of ˜10 cm long. The tubing was connected to a syringe. After the cannulation, a droplet of super glue was used to secure the position of the needle on the tail. In the nanosphere experiment, a 50-μl suspension of nanospheres in PBS was injected over an approximately 5-second period. At 5 min after the first injection, we replaced the syringe with a new one containing the nanospheres suspended in the poloxamer solution and injected the suspension into the same tail vein. During and after both injections, the video of nanosphere movement in the tail was recorded continuously under an INTRAVITAL™ fluorescence microscope (MPS, Carl Zeiss, Hanover, Md., U.S.A.) equipped with a rhodamine filter set and a color video camera (Carl Zeiss ZVS-3C75DE), which was connected to a videocassette recorder (Sony S-HVS, Sony Corporation of America, N.Y., N.Y., U.S.A.). After the experiment, individual images at different time points were captured from the video, using the IMAGE-PRO PLUS® software (Media Cybernetics, Inc., Sliver Spring, Md., U.S.A.). In the AdCMVLuc experiment, the procedure for the tail vein injection was the same as that described in the present Example. The dose of injection was 1.0×10⁸ pfu/mouse. At 24 hrs after the injection of AdCMVLuc suspension with or without poloxamer, luciferase expression was examined in mice using the Xenogen IVIS (Xenogen Corp.). The nanosphere and AdCMVLuc experiments were repeated three times and the typical results are shown in the present Example and associated Figures.

Statistical analysis. The Mann-Whitney U test was used to compare differences between two unpaired groups. The difference was considered to be significant if the p value was less than 0.05.

### Results and Discussion for Example 6

The viscosity of the poloxamer solution (21%, w/w) depended on temperature and shear rate. When the temperature was changed from 4° C. to 37° C., the viscosity could be increased by several orders of magnitude, depending on the shear rate. At the shear rate of 7.5 see, the viscosity was increased from 41 to 4200 cP within a few seconds.

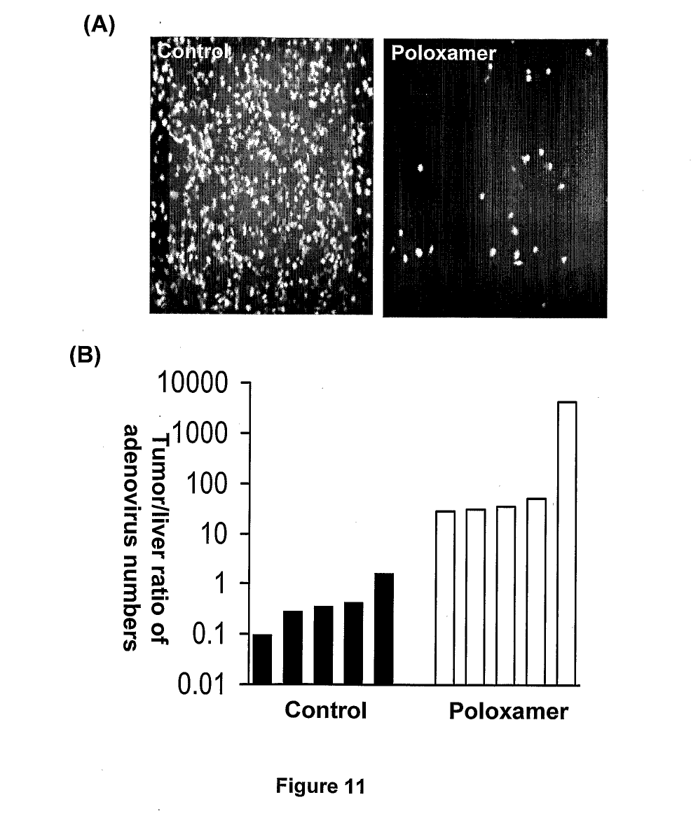

The rapid increase in the viscosity allows the poloxamer solution to retain viral vectors in tumors during and after intratumoral infusion. To directly demonstrate this, AdCMVEGFP suspended in the poloxamer solution was infused into 4T1 tumors. Immediately after the infusion, it was observed that the poloxamer solution could significantly reduce the number of viral vectors, isolated from the blood, that could transfect 293 cells in vitro (see FIG. 11A). Quantitatively, it was observed that the poloxamer solution could reduce the copy number of AdCMVEGFP in the liver and increase the copy number of AdCMVEGFP in the tumor at 10 min after the infusion. The ratios of copy numbers between tumor and liver in individual animals are shown in FIG. 7B. The lowest ratio in the poloxamer group was 28 whereas the highest ratio in the control group was only 1.6. The median ratios were 35.1 and 0.35 in the poloxamer and control groups, respectively.

It was also observed that two mice in the control group died within half an hour after the intratumoral infusion of AdCMVEGFP, but mice in the poloxamer group did not show any problem after the infusion. The death was likely due to acute immune response to viral vectors escaped from the tumor during and after the intratumoral infusion (Varnayski et al., 2005; Muruve, 2004). These results provide direct evidence showing that the poloxamer solution could reduce the systemic toxicity caused by the virus dissemination.

In addition to the effects of poloxamer solution on virus dissemination, the effects of the same solution on transgene expression in normal and tumor tissues was examined at 24 hrs after the intratumoral infusion of AdCMVEGFP or AdCMVLuc. It was observed that EGFP expression in the liver was minimal in the poloxamer group but strong in the control group (FIG. 12A). In the tumor, the opposite was observed, i.e., EGFP expression in the poloxamer group was stronger than that in the control group (FIG. 12A). Luciferase expression, indicated by the bioluminescence, is shown in FIGS. 12B through 12D. Its distribution in mice indicated that the poloxamer solution could significantly reduce the transgene expression in the liver and increase the transgene expression in the tumor (FIGS. 12B and 12C). Consequently, the poloxamer solution could increase the tumor/liver ratio of transgene expressions by 12- to 275-fold (FIG. 12D). These results were consistent with those in the EGFP experiment shown in FIG. 12A. Without wishing to be bound by a particular theory of operation, the mechanism of reduction in virus dissemination was likely to be that the poloxamer solution blocked the convection of viral vectors in the interstitial space and the lumen of microvessels in the vicinity of the infusion site, since it was demonstrated that infusion-induced convective transport is the mechanism of dissemination (Wang et al., 2003; Examples 1-4).

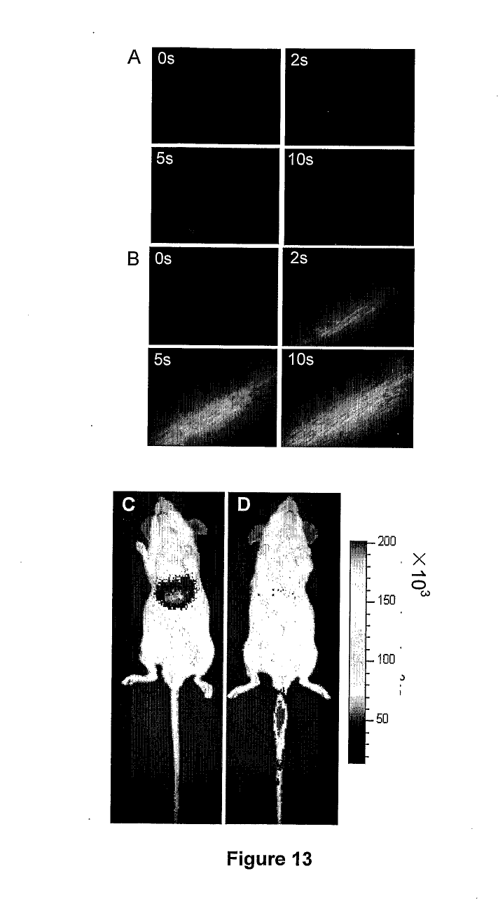

To directly demonstrate that the poloxamer solution could block convective transport in blood vessels, a suspension of polystyrene nanospheres was injected into a mouse tail vein. Without poloxamer, it was observed that the nanospheres disappeared within a few seconds from the tail vein after the injection (FIG. 13A). Although the nanospheres could circulate in mice, they were significantly diluted by the blood and most of them were likely to be rapidly taken up by normal organs. Thus, the nanospheres could be observed only during the first pass through the tail vein. It was also observed that the width of the fluorescence distribution in the tail was larger than the diameter of the vein. This was potentially caused by light scattering in tissues and unlikely to be due to the accumulation of nanospheres in the perivascular regions because, if the latter happened, the nanospheres would be cleared slowly from the tail after the injection. In contrast, nanospheres suspended in the poloxamer solution stayed in the tail after the injection (FIG. 13B). They could accumulate both within the lumen of the tail vein and in the surrounding interstitial space.

Flow resistance in the tail vein increased rapidly with time during the injection due to the in situ gelation of poloxamer and is likely a reason for these observations. The gel blocked the flow and led to a rapid increase in microvascular pressure when the injection was continued. The pressure increase could cause rupture of the vein, which allowed the nanospheres to leak out and accumulate in the interstitial space. Therefore, the fluorescence distribution in the poloxamer group was much wider than that in the control group. It was unlikely that the rupture occurred during the tail vein cannulation since the same vessel was used for both injections and no significant leak out of nanospheres was observed when the suspension without poloxamer was injected (see FIG. 13A). During the intratumoral infusion, the pressure-induced rupture of tumor microvessels was not an issue since the interstitial fluid pressure was always higher than or equal to the microvascular pressure. In this case, tumor microvessels could only be compressed. Vessel compression might be another mechanism of reduction in virus dissemination.

In addition to the nanospheres, a suspension of AdCMVLuc was injected into the tail vein. Without poloxamer, it was observed that the luciferase expression was high in the liver and minimal in the tail at 24 hr after the injection (FIG. 13C). With poloxamer, the opposite was observed, i.e., the luciferase expression was high in the tail and minimal in the liver (FIG. 13D). These results were consistent with the distribution of nanospheres in the tail shown in FIG. 13B, indicating that the viral vectors accumulated both within the lumen of the tail vein and in the surrounding interstitial space. As a result, the viral vectors might have transfected vascular endothelial cells and cells in perivascular regions (FIG. 13D).

One question addressed by the present Example was whether poloxamer would chemically change the ability of adenoviral vectors to transfect cells. To answer this question, AdCMVEGFP was mixed with PBS or a diluted poloxamer solution (0.1% w/w) and the mixture used to treat cultured 4T1 cells. It was observed that the percentage of EGFP positive cells was not affected by the presence of poloxamer, and thus poloxamer does not appear to affect vector transfection of target cells.

A potential adverse effect of the poloxamer solution is that microclots could escape from the tumor and accumulate in normal organs. However, this effect is likely to be insignificant since Poloxamer 407 has been previously used as a temporary vascular occlusion agent (Raymond et al., 2004). Although small fragments of clots may detach and escape from the occluded vessels, especially the large veins (i.e., iliac veins and vena cava), they will likely accumulate in the lungs and be rapidly dissolved there (Raymond et al., 2004). The rate of disolution is much faster than that of large clots formed in the lung after a direct injection into the pulmonary artery, because the rate of dissolution decreases with increasing size of clots. In the present Example, poloxamer 407 was infused into tumor tissues. Thus, clots could be formed only in tumor microvessels and the interstitial space so that they were much smaller than those formed in the large vessels mentioned above and therefore will likely rapidly dissolve if some fragments escape from the tumor. Another related issue was the leak out of poloxamer solution from the tumor before viscosity increased. In this case, the polymer solution would be significantly diluted by the blood and hence unlikely to form clots in normal tissues.

In summary, the present Example provides data showing that the delivery method of the presently disclosed subject matter can significantly increase virus copy number and transgene expression a target tissue and decrease systemic dissemination, thereby reducing copy number in the liver in intratumoral infusion-mediated gene delivery. This improvement is beneficial for enhancing the efficacy/toxicity ratio in viral gene therapy. Although the present Example tested adenoviral vectors, it could also be implemented to deliver other therapeutic agents in solid tumors because the mechanisms of passive transport are the same for all agents. Furthermore, the same method can be useful for delivering viral vectors in other target tissues where systemic dissemination is a significant problem.

### REFERENCES

The references listed below, as well as all references cited in the specification, are incorporated herein by reference to the extent that they supplement, explain, provide a background for, or teach methodology, techniques, and/or compositions employed herein.
Adachi, Y., Reynolds, P. N., Yamamoto, M., Wang, M., Takayama, K., Matsubara, S., Muramatsu, T. & Curiel, D. T. (2001) A midkine promoter-based conditionally replicative adenovirus for treatment of pediatric solid tumors and bone marrow tumor purging. Cancer Res, 61, 7882-8.
Adelman et al. (1983) DNA 2:183.
Albini A, Marchisone C, Del Grosso F, Benelli R, Masiello L, Tacchetti C, Bono M, Ferrantini M, Rozera C, Truini M, Belardelli F, Santi L & Noonan D M (2000) Inhibition of angiogenesis and vascular tumor growth by interferon-producing cells: A gene therapy approach. Am J Pathol 156:1381-1393.
Alemany, R., Suzuki, K. & Curiel, D. T. (2000) Blood clearance rates of adenovirus type 5 in mice. J Gen Virol, 81, 2605-9.
Ausubel et al. (1992) Current Protocols in Molecular Biology, John Wylie and Sons, Inc., New York, N.Y., United States of America.
Blair G E (2004) Adenoviral vectors, breaking a barrier to gene therapy? Gene Ther. 11: 229-30.
Boucher Y, Brekken C, Netti P A, Baxter L T, Jain R K (1998) Intratumoral infusion of fluid: estimation of hydraulic conductivity and implications for the delivery of therapeutic agents. Br J Cancer 78: 1442-8.
Bramson, J. L., Hitt, M., Gauldie, J. & Graham, F. L. (1997) Pre-existing immunity to adenovirus does not prevent tumor regression following intratumoral administration of a vector expressing IL-12 but inhibits virus dissemination. Gene Ther, 4, 1069-76.
Bristol J A, Shirley P, Idamakanti N, Kaleko M, Connelly S (2000) In vivo dose threshold effect of adenovirus-mediated factor VIII gene therapy in hemophiliac mice. Mol Ther 2: 223-32.
Brunetti-Pierri, N., D. J. Palmer, A. L. Beaudet, K. D. Carey, M. Finegold, and P. Ng (2004) Acute toxicity after high-dose systemic injection of helper-dependent adenoviral vectors into nonhuman primates. Hum Gene Ther 15:35-46.
Carpizo D & Iruela-Arispe M L (2000) Endogenous regulators of angiogenesis—emphasis on proteins with thrombospondin—type I motifs. Cancer Metastasis Rev 19:159-165.
Cheng (1996) Human Gene Therapy 7:275.
Cichon, G., Boeckh-Herwig, S., Kuemin, D., Hoffmann, C., Schmidt, H. H., Wehnes, E., Haensch, W., Schneider, U., Eckhardt, U., Burger, R. & Pring-Akerblom, P. (2003) Titer determination of Ad5 in blood: a cautionary note. Gene Ther, 10, 1012-7.
Clapp C, Martial J A, Guzman R C, Rentier-Delure F & Weiner R I (1993) The 16-kilodalton N-terminal fragment of human prolactin is a potent inhibitor of angiogenesis. Endocrinology 133:1292-1299.
Dameron K M, Volpert O V, Tainsky M A & Bouck N (1994) Control of angiogenesis in fibroblasts by p53 regulation of thrombospondin-1. Science 265:1582-1584.
DeWeese, T. L., van der Poel, H., Li, S., Mikhak, B., Drew, R., Goemann, M., Hamper, U., DeJong, R., Detorie, N., Rodriguez, R., Haulk, T., DeMarzo, A. M., Piantadosi, S., Yu, D. C., Chen, Y., Henderson, D. R., Carducci, M. A., Nelson, W. G. & Simons, J. W. (2001) A phase I trial of CV706, a replication-competent, PSA selective oncolytic adenovirus, for the treatment of locally recurrent prostate cancer following radiation therapy. Cancer Res, 61, 7464-72.
Dillehay L E. (1997) Decreasing resistance during fast infusion of a subcutaneous tumor. Anticancer Res 17: 461-6.
Eijan A M, Davel L, Oisgold-Daga S & de Lustig E S (1991) Modulation of tumor-induced angiogenesis by proteins of extracellular matrix. Mol Biother 3:38-40.
European Patent No. 0 439 095.
Flickinger & Trost (1976) Eu. J. Cancer 12(2):159-60.
Glover ed (1985) DNA Cloning: A Practical Approach, MRL Press, Ltd., Oxford, United Kingdom.
Goldman et al. (1997) Nat Biotechnol 15:462.
Graham F L, Prevec L. (1991) Manipulation of adenovirus vectors. In: Murray E J, ed. Gene transfer and expression protocols. Clifton: Humana Press; p. 109-28.
Graham, F. L. & Prevec, L. (1995) Methods for construction of adenovirus vectors. Mol Biotechnol, 3, 207-20.
Habib et al. (1999) Human Gene Therapy 12:2019-2034.
Hackett, N. R., E I Sawy, T., Lee, L. Y., Silva, I., O'Leary, J., Rosengart, T. K. & Crystal, R. G. (2000) Use of quantitative TaqMan real-time PCR to track the time-dependent distribution of gene transfer vectors in vivo. Mol Ther, 2, 649-56.
Haegel-Kronenberger H et al. (2004) Inhibition of costimulation allows for repeated systemic administration of adenoviral vector in rhesus monkeys. Gene Ther. 11: 241-52.
Hecht E, Mortensen K, Gradzielski M, Hoffmann H (1995) Interaction of Aba Block-Copolymers with Ionic Surfactants—Influence on Micellization and Gelation. J Phys Chem-Us 99: 4866-74.
Herz, J. & Gerard, R. D. (1993) Adenovirus-mediated transfer of low density lipoprotein receptor gene acutely accelerates cholesterol clearance in normal mice. Proc Natl Acad Sci USA, 90, 2812-6.
Hobbs, S. K., Monsky, W., Yuan, F., Roberts, W. G., Griffith, L., Torchillin, V. P. & Jain, R. K. (1998) Regulation of transport pathways in tumor vessels: Role of tumor type and microenvironment. Proc Nat Acad Sci USA, 95, 4607-4612.
Ingber D, Fujita T, Kishimoto S, Sudo K, Kanamaru T, Brem H & Folkman J (1990) Synthetic analogues of fumagillin that inhibit angiogenesis and suppress tumour growth. Nature 348:555-557.
Jain, R. K. (1997) Delivery of molecular and cellular medicine to solid tumors. Microcirculation, 4, 1-23.
Jooss K, Turka L A, & Wilson J M (1998) Blunting of immune responses to adenoviral vectors in mouse liver and lung with CTLA4Ig. Gene Ther 5: 309-19.
Kirpotin et al. (1997) Biochemistry 36:66.
Kosfeld M D & Frazier W A (1993) Identification of a new cell adhesion motif in two homologous peptides from the COOH-terminal cell binding domain of human thrombospondin. J Biol Chem 268:8808-8814.
Lee, Y. T. (1983) Breast carcinoma: pattern of metastasis at autopsy. J Surg Oncol, 23, 175-80.
Lohr, F., Huang, Q., Hu, K., Dewhirst, M. W. & Li, C. Y. (2001) Systemic vector leakage and transgene expression by intratumorally injected recombinant adenovirus vectors. Clin Cancer Res, 7, 3625-8.
Lu, Y., Carraher, J., Zhang, Y., Armstrong, J., Lerner, J., Rogers, W. P. & Steiner, M. S. (1999) Delivery of adenoviral vectors to the prostate for gene therapy. Cancer Gene Ther, 6, 64-72.
Maione T E, Gray G S, Petro J, Hunt A J, Donner A L, Bauer S I, Carson H F & Sharpe R J (1990) Inhibition of angiogenesis by recombinant human platelet factor-4 and related peptides. Science 247:77-79.
Marin et al. (1997) Mol Med Today 3:396.
Marshall, E. (1999) Gene therapy death prompts review of adenovirus vector. Science 286:2244-2245.
McGuire S, Yuan F (2001) Quantitative analysis of intratumoral infusion of color molecules. Am J Physiol Heart Circ Physiol 281: H715-21.
Muruve D A (2004) The innate immune response to adenovirus vectors. Hum Gene Ther 15: 1157-66.
Nabel (1997) “Vectors for Gene Therapy” in Current Protocols in Human Genetics on CD-ROM, John Wiley & Sons, New York, N.Y.
Nasu, Y., Bangma, C. H., Hull, G. W., Lee, H. M., Hu, J., Wang, J., McCurdy, M. A., Shimura, S., Yang, G., Timme, T. L. & Thompson, T. C. (1999) Adenovirus-mediated interleukin-12 gene therapy for prostate cancer: suppression of orthotopic tumor growth and pre-established lung metastases in an orthotopic model. Gene Ther, 6, 338-49.
Nemunaitis, J., Khuri, F., Ganly, I., Arseneau, J., Posner, M., Vokes, E., Kuhn, J., McCarty, T., Landers, S., Blackburn, A., Romel, L., Randlev, B., Kaye, S. & Kirn, D. (2001) Phase II trial of intratumoral administration of ONYX-015, a replication-selective adenovirus, in patients with refractory head and neck cancer. J Clin Oncol, 19, 289-98.
Neri et al. (1997) Nat BioTechnology 15:1271.
Netti, P. A., Baxter, L. T., Boucher, Y., Skalak, R. & Jain, R. K. (1995) Time-dependent behavior of interstitial fluid pressure in solid tumors: implications for drug delivery. Cancer Res, 55, 5451-5458.
O'Reilly M S, Holmgren L, Shing Y, Chen C, Rosenthal R A, Moses M, Lane W S, Cao Y, Sage E H & Folkman J (1994) Angiostatin: a novel angiogenesis inhibitor that mediates the suppression of metastases by a Lewis lung carcinoma. Cell 79:315-328.
O'Reilly M S, Boehm T, Shing Y, Fukai N, Vasios G, Lane W S, Flynn E, Birkhead J R, Olsen B R & Folkman J (1997) Endostatin: an endogenous inhibitor of angiogenesis and tumor growth. Cell 88:277-285.
Paielli, D. L., Wing, M. S., Rogulski, K. R., Gilbert, J. D., Kolozsvary, A., Kim, J. H., Hughes, J., Schnell, M., Thompson, T. & Freytag, S. O. (2000) Evaluation of the biodistribution, persistence, toxicity, and potential of germ-line transmission of a replication-competent human adenovirus following intraprostatic administration in the mouse. Mol Ther, 1, 263-74.
Park et al. (1997) Proc Am Ass Canc Res 38:342.
Pasqualini et al. (1997) Nat Biotechnology 15:542.
Penuelas I et al. (2005) Positron emission tomography imaging of adenoviral-mediated transgene expression in liver cancer patients. Gastroenterology 128: 1787-95.
Raper S E, Yudkoff M, Chirmule N, et al. (2002) A pilot study of in vivo liver-directed gene transfer with an adenoviral vector in partial ornithine transcarbamylase deficiency. Hum Gene Ther 13: 163-75.
Raymond J, Metcalfe A, Salazkin I, Schwarz A (2004) Temporary vascular occlusion with poloxamer 407. Biomaterials 25: 3983-9.
Sakamoto N, Iwahana M, Tanaka N G & Osada Y (1991) Inhibition of angiogenesis and tumor growth by a synthetic laminin peptide, CDPGYIGSR-NH₂. Cancer Res 51:903-906.
Sambrook & Russell, 2001; Molecular Cloning, Third Edition, Cold Spring Harbor Laboratory Press, Cold Spring Harbor, N.Y., United States of America.
Sauthoff, H., Hu, J., Maca, C., Goldman, M., Heitner, S., Yee, H., Pipiya, T., Rom, W. N. & Hay, J. G. (2003) Intratumoral spread of wild-type adenovirus is limited after local injection of human xenograft tumors: virus persists and spreads systemically at late time points. Hum Gene Ther, 14, 425-33.
Silhavy et al. (1984) Experiments with Gene Fusions, Cold Spring Harbor Laboratory Press, Cold Spring Harbor, N.Y., United States of America.
Tang D C, Johnston S A, Carbone D P (1994) Butyrate-inducible and tumor-restricted gene expression by adenovirus vectors. Cancer Gene Ther 1: 15-20.
Tjuvajev, J. G., Chen, S. H., Joshi, A., Joshi, R., Guo, Z. S., Balatoni, J., Ballon, D., Koutcher, J., Finn, R., Woo, S. L. & Blasberg, R. G. (1999) Imaging adenoviral-mediated herpes virus thymidine kinase gene transfer and expression in vivo. Cancer Res, 59, 5186-93.
Toloza, E. M., Hunt, K., Swisher, S., McBride, W., Lau, R., Pang, S., Rhoades, K., Drake, T., Belldegrun, A., Glaspy, J. & Economou, J. S. (1996) In vivo cancer gene therapy with a recombinant interleukin-2 adenovirus vector. Cancer Gene Ther, 3, 11-7.
Tolsma S S, Volpert O V, Good D J, Frazier W A, Polyerini P J & Bouck N (1993) Peptides derived from two separate domains of the matrix protein thrombospondin-1 have anti-angiogenic activity. J Cell Biol 122:497-511.
UKCCCR. (1998) United Kingdom Co-ordinating Committee on Cancer Research (UKCCCR) Guidelines for the Welfare of Animals in Experimental Neoplasia (Second Edition) Br J Cancer, 77, 1-10.
U.S. Pat. No. 4,551,482.
U.S. Pat. No. 5,714,166.
U.S. Pat. No. 5,574,172.
U.S. Pat. No. 6,071,890.
Varnayski A N, Calcedo R, Bove M, Gao G, Wilson J M (2005) Evaluation of toxicity from high-dose systemic administration of recombinant adenovirus vector in vector-naive and pre-immunized mice. Gene Ther 12: 427-36.
Voest E E, Kenyon B M, O'Reilly M S, Truitt G, D'Amato R J & Folkman J (1995) Inhibition of angiogenesis in vivo by interleukin 12. J Natl Cancer Inst 87:581-586.
Vogel & Muller-Eberhard (1981) Anal. Biochem 118(2):262-268.
Wang P & Johnston T P. (1991) Kinetics of Sol-to-Gel Transition for Poloxamer Polyols. J Appl Polym Sci 43: 283-92.
Wang, Y., Hu, J. K., Krol, A., Li, Y. P., Li, C. Y. & Yuan, F. (2003) Systemic dissemination of viral vectors during intratumoral injection. Mol Cancer Ther, 2, 1233-42.
Wang, Y., S. Liu, C. Y. Li, and F. Yuan (2005a) A novel method for viral gene delivery in solid tumors. Cancer Res 65:7541-7545.
Wang, Y., Z. Yang, S. Liu, T. Kon, A. Krol, C. Y. Li, and F. Yuan (2005b) Characterization of systemic dissemination of nonreplicating adenoviral vectors from tumours in local gene delivery. Br J Cancer 92:1414-1420.
Wang Y, Yuan F. (2006) Delivery of viral vectors to tumor cells: Extracellular transport, systemic distribution, and strategies for improvement. Annals Biomed Eng; in press.
Woltering E A, Barrie R, O'Dorisio T M, Arce D, Ure T, Cramer A, Holmes D, Robertson J & Fassler J (1991) Somatostatin analogues inhibit angiogenesis in the chick chorioallantoic membrane. J Surg Res 50:245-251.
Worgall, S., Wolff, G., Falck-Pedersen, E. & Crystal, R. G. (1997) Innate immune mechanisms dominate elimination of adenoviral vectors following in vivo administration. Hum Gene Ther, 8, 37-44.
Yuan, F. (1998) Transvascular drug delivery in solid tumors. Semin Radiat Oncol, 8, 164-75.
Zaiss A K & Muruve D A (2005) Immune responses to adeno-associated virus vectors. Curr Gene Ther 5: 323-31.
Zhang R, Straus F H, DeGroot L J (1999) Effective genetic therapy of established medullary thyroid carcinomas with murine interleukin-2: dissemination and cytotoxicity studies in a rat tumor model. Endocrinology 140: 2152-8.

It will be understood that various details of the presently disclosed subject matter may be changed without departing from the scope of the present subject matter. Furthermore, the foregoing description is for the purpose of illustration only, and not for the purpose of limitation.

What is claimed is:

1. A method for reducing leakage of a delivery formulation from a target tissue via a damaged blood vessel during and after administration of the delivery formulation to the target tissue, the method comprising:
(a) providing a delivery formulation comprising a blocking agent and one or more vectors encoding one or more polypeptides, wherein the delivery formulation is in a liquid form; and
(b) administering the delivery formulation to a target tissue in a subject, wherein the administering results in the viscosity of the blocking agent increasing to at least about 100 cP, whereby leakage of the delivery formulation from the target tissue either during or after administration of the delivery formulation is reduced.

2. The method of claim 1, wherein the vector is a viral vector.

3. The method of claim 2, wherein the viral vector is selected from the group consisting of a retrovirus vector, a pseudotyped retrovirus vector, an adenovirus vector, an adeno-associated virus vector, a herpes virus vector, a vaccinia virus vector, a Semliki Forest virus vector, and a baculovirus vector.

4. The method of claim 1, wherein the vector is a non-viral vector.

5. The method of claim 4, wherein the non-viral vector is selected from the group consisting of plasmids, bacteria, water-oil emulsions, polyethylene imines, dendrimers, micelles, microcapsules, liposomes, and cationic lipids.

6. The method of claim 1, wherein the polypeptide is a therapeutic polypeptide selected from the group consisting of reporter molecules, immunostimulatory molecules, enzymes that can convert prodrugs to drugs, tumor suppressor gene products, tumor antigens, apoptosis mediators, toxins, and anti-angiogenic factors.

7. The method of claim 6, wherein the immunostimulatory molecule is a cytokine.

8. The method of claim 1, wherein the increase in viscosity of the blocking agent does not result in a significant trapping of the vector in the delivery formulation or a significant reduction in the efficiency of the vector to infect the target tissue.

9. The method of claim 1, wherein the viscosity of the blocking agent is less than about 20 cP before administration, and increases to at least about 100 cP after administration.

10. The method of claim 9, wherein the viscosity of the blocking agent increases to at least about 100 cP within about 1 sec after administration.

11. The method of claim 10, wherein the viscosity of the blocking agent increases to a value with a range of about 200 cP to about 100,000 cP after administration.

12. The method of claim 1, wherein the blocking agent comprises a poloxamer-containing formulation.

13. The method of claim 12, wherein the poloxamer is present in the poloxamer-containing formulation in a concentration ranging from about 10% to about 40% by weight.

14. The method of claim 1, wherein the blocking agent comprises an alginate-containing formulation.

15. The method of claim 14, wherein the alginate is present in the alginate-containing formulation in a concentration ranging from about 0.1% to about 8% by weight.

16. A method for decreasing systemic toxicity of a delivery formulation, the method comprising:
(a) providing a delivery formulation comprising a blocking agent and one or more vectors encoding one or more polypeptides, wherein the delivery formulation is in a liquid form; and
(b) administering the delivery formulation to a target tissue in a subject, wherein the administering results in the viscosity of the blocking agent increasing to at least about 100 cP, whereby systemic toxicity of the delivery formulation is decreased.

17. The method of claim 16, wherein the vector is a viral vector.

18. The method of claim 17, wherein the viral vector is selected from the group consisting of a retrovirus vector, a pseudotyped retrovirus vector, an adenovirus vector, an adeno-associated virus vector, a herpes virus vector, a vaccinia virus vector, a Semliki Forest virus vector, and a baculovirus vector.

19. The method of claim 16, wherein the vector is a non-viral vector.

20. The method of claim 19, wherein the non-viral vector is selected from the group consisting of plasmids, bacteria, water-oil emulsions, polyethylene imines, dendrimers, micelles, microcapsules, liposomes, and cationic lipids.

21. The method of claim 16, wherein the peptide is a therapeutic polypeptide selected from the group consisting of reporter molecules, immunostimulatory molecules, enzymes that can convert prodrugs to drugs, tumor suppressor gene products, tumor antigens, apoptosis mediators, toxins, and anti-angiogenic factors.

22. The method of claim 21, wherein the immunostimulatory molecule is a cytokine.

23. The method of claim 16, wherein the increase in viscosity of the blocking agent does not result in a significant trapping of the vector in the delivery formulation or a significant reduction in the efficiency of the vector to infect the target tissue.

24. The method of claim 16, wherein the viscosity of the blocking agent is less than about 20 cP before administration, and increases to at least about 100 cP after administration.

25. The method of claim 24, wherein the viscosity of the blocking agent increases to at least about 100 cP within about 1 sec after administration.

26. The method of claim 25, wherein the viscosity of the blocking agent increases to a value with a range of about 200 cP to about 100,000 cP after administration.

27. The method of claim 16, wherein the blocking agent comprises a poloxamer-containing formulation.

28. The method of claim 27, wherein the poloxamer is present in the poloxamer-containing formulation in a concentration ranging from about 10% to about 40% by weight.

29. The method of claim 16, wherein the blocking agent comprises an alginate-containing formulation.

30. The method of claim 29, wherein the alginate is present in the alginate-containing formulation in a concentration ranging from about 0.1% to about 8% by weight.

31. A method for reducing immune system detection in a subject of a delivery formulation, the method comprising:
(a) providing a delivery formulation comprising a blocking agent and one or more vectors encoding one or more polypeptides, wherein the delivery formulation is in a liquid form; and
(b) administering the delivery formulation to a target tissue in a subject, wherein the administering results in the viscosity of the blocking agent increasing to at least about 100 cP, whereby immune system detection of the delivery formulation in the subject is decreased.

32. The method of claim 31, wherein the vector is a viral vector.

33. The method of claim 32, wherein the vector is a viral vector selected from the group consisting of a retrovirus vector, a pseudotyped retrovirus vector, an adenovirus vector, an adeno-associated virus vector, a herpes virus vector, a vaccinia virus vector, a Semliki Forest virus vector, and a baculovirus vector.

34. The method of claim 31, wherein the vector is a non-viral vector.

35. The method of claim 34, wherein the non-viral vector is selected from the group consisting of plasmids, bacteria, water-oil emulsions, polyethylene imines, dendrimers, micelles, microcapsules, liposomes, and cationic lipids.

36. The method of claim 31, wherein the peptide is a therapeutic polypeptide selected from the group consisting of reporter molecules, immunostimulatory molecules, enzymes that can convert prodrugs to drugs, tumor suppressor gene products, tumor antigens, apoptosis mediators, toxins, and anti-angiogenic factors.

37. The method of claim 36, wherein the immunostimulatory molecule is a cytokine.

38. The method of claim 31, wherein the increase in viscosity of the blocking agent does not result in a significant trapping of the vector in the delivery formulation or a significant reduction in the efficiency of the vector to infect the target tissue.

39. The method of claim 31, wherein the viscosity of the blocking agent is less than about 20 cP before administration, and increases to at least about 100 cP after administration.

40. The method of claim 39, wherein the viscosity of the blocking agent increases to at least about 100 cP within about 1 sec after administration.

41. The method of claim 40, wherein the viscosity of the blocking agent increases to a value with a range of about 200 cP to about 100,000 cP after administration.

42. The method of claim 31, wherein the blocking agent comprises a poloxamer-containing formulation.

43. The method of claim 42, wherein the poloxamer is present in the poloxamer-containing formulation in a concentration ranging from about 10% to about 40% by weight.

44. The method of claim 31, wherein the blocking agent comprises an alginate-containing formulation.

45. The method of claim 44, wherein the alginate is present in the alginate-containing formulation in a concentration ranging from about 0.1% to about 8% by weight.

46. A method for increasing efficiency of vector delivery to a target tissue, the method comprising:
(a) providing a delivery formulation comprising a blocking agent and one or more vectors encoding one or more polypeptides, wherein the delivery formulation is in a liquid form; and
(b) administering the delivery formulation to a target tissue in a subject, wherein the administering results in the viscosity of the blocking agent increasing to at least about 100 cP, wherein the increase in viscosity of the blocking agent reduces leakage of the vector from the target tissue to thereby increase the efficiency of vector delivery to the target tissue.

47. The method of claim 46, wherein the increased efficiency of vector delivery results in an increase in the expression of the therapeutic polypeptide in the target tissue of at least about 2- to about 4-fold when compared to the expression of the therapeutic polypeptide in the target tissue when an equivalent dosage of the viral vector is administered in the absence of the blocking agent.

48. The method of claim 46, wherein the vector is a viral vector.

49. The method of claim 48, wherein the viral vector is selected from the group consisting of a retrovirus vector, a pseudotyped retrovirus vector, an adenovirus vector, an adeno-associated virus vector, a herpes virus vector, a vaccinia virus vector, a Semliki Forest virus vector, and a baculovirus vector.

50. The method of claim 46, wherein the vector is a non-viral vector.

51. The method of claim 50, wherein the non-viral vector is selected from the group consisting of plasmids, bacteria, water-oil emulsions, polyethylene imines, dendrimers, micelles, microcapsules, liposomes, and cationic lipids.

52. The method of claim 46, wherein the peptide is a therapeutic polypeptide selected from the group consisting of reporter molecules, immunostimulatory molecules, enzymes that can convert prodrugs to drugs, tumor suppressor gene products, tumor antigens, apoptosis mediators, toxins, and anti-angiogenic factors.

53. The method of claim 52, wherein the immunostimulatory molecule is a cytokine.

54. The method of claim 46, wherein the increase in viscosity of blocking agent does not result in a significant trapping of the vector in the delivery formulation or a significant reduction in the efficiency of the vector to infect the target tissue.

55. The method of claim 46, wherein the viscosity of the blocking agent is less than about 20 cP before administration, and increases to at least about 100 cP after administration.

56. The method of claim 55, wherein the viscosity of the blocking agent increases to at least about 100 cP within about 1 sec after administration.

57. The method of claim 56, wherein the viscosity of the blocking agent increases to a value with a range of about 200 cP to about 100,000 cP after administration.

58. The method of claim 46, wherein the blocking agent comprises a poloxamer-containing formulation.

59. The method of claim 58, wherein the poloxamer is present in the poloxamer-containing formulation in a concentration ranging from about 10% to about 40% by weight.

60. The method of claim 46, wherein the blocking agent comprises an alginate-containing formulation.

61. The method of claim 60, wherein the alginate is present in the alginate-containing formulation in a concentration ranging from about 0.1% to about 8% by weight.


SEQUENCE LISTING (3 sequences)

SEQ ID NO: 1 - DNA, 21 bp, Artificial sequence
  Artificially synthesized forward nucleic acid primer sequence for PCR amplification of the adenovirus E4 region.
tgacacgcatactcggagcta

SEQ ID NO: 2 - DNA, 20 bp, Artificial sequence
  Artificially synthesized reverse nucleic acid primer sequences for PCR amplification of the adenovirus E4 region.
tttgagcagcaccttgcatt

SEQ ID NO: 3 - DNA, 21 bp, Artificial sequence
  Artificially synthesized nucleic acid sequence probe for capture of adenovirus nucleic acids.
cgccgcccatgcaacaagctt